(19) United States
(12) Patent Application Publication
(10) Pub. No.: US 2026/0208165 A1
(43) Pub. Date: Jul. 23, 2026

(54) HETEROGENEOUS BIMETALLIC CATALYST, METHOD FOR PRODUCING SAME AND USE OF SAME

(71) Applicant: FAIRBRICS, CLICHY (FR)

(72) Inventors: Ibrahim ABDELLAH, Montigny Les Metz (FR); Ali NASRALLAH, Laval (CA); Tawfiq NASR ALLAH, Paris (FR)

(73) Assignee: FAIRBRICS, CLICHY (FR)

(21) Appl. No.: 19/138,243

(22) PCT Filed: Dec. 13, 2023
(86) PCT No.: PCT/EP2023/085706

§ 371 Date: Jun. 12, 2025

(30) Foreign Application Priority Data
Dec. 13, 2022 (FR) 2213276

(51) Int. Cl. B01J 23/75; B01J 21/06; B01J 35/30; B01J 35/40; B01J 35/51; B01J 35/61; B01J 37/02; B01J 37/08; C07C 29/149

(52) U.S. Cl. CPC B01J 23/75; B01J 21/066; B01J 35/394; B01J 35/40; B01J 35/51; B01J 35/612; B01J 35/613; B01J 37/0201; B01J 37/0236; B01J 37/088; C07C 29/149

(57) ABSTRACT

A new supported bimetallic catalyst including copper and cobalt on a zirconium dioxide support, of formula CoCu/ZrO₂, and the use of the catalyst in the implementation of a method of preparation of ethylene glycol from an oxalate compound by a hydrogenation reaction of the oxalate compound by hydrogen (H₂) to obtain ethylene glycol. Also, the use of the catalyst in the depolymerization of biomass.

The present invention relates to a novel heterogeneous bimetallic catalyst, its method of preparation and its use, in particular for the synthesis of ethylene glycol from oxalate hydrogenation.

Ethylene glycol is an important material in the chemical industry, enabling various applications as antifreeze or refrigerant, but especially in particular the production of textile fibers and polyester resins. Ethylene glycol can be used as a monomer in the manufacture of polyester, and in particular PET (Polyethylene terephthalate) in the presence of terephthalic acid. PET is a polymer widely used in the textile industry, which today is mainly produced from petroleum-based products that generate a large amount of greenhouse gases, including carbon dioxide.

A typical method of manufacturing ethylene glycol is carried out from naphtha and is shown below:

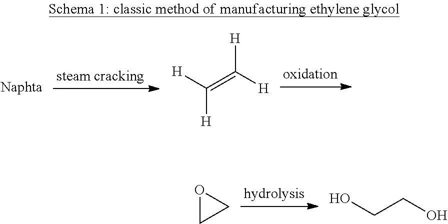

The recovery of carbon dioxide CO₂ is a major challenge to reduce greenhouse gas emissions. Another route of ethylene glycol synthesis is the hydrogenation of oxalates or oxamides. Oxalates are raw materials with high added value in the chemical industry. They are used on a large scale to produce various dyes, drugs, solvents, extractants, and various intermediates in the fine chemical industry.

Thus, CO₂ consumption can be achieved during the synthesis of oxalates, which are precursors to produce ethylene glycol via a catalysed hydrogenation reaction with transition metals, as homogeneous or heterogeneous catalysts, as schematized below:

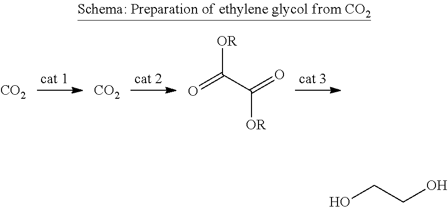

A homogeneous transition metal-based catalyst is a catalyst that is soluble in the solvent of the reaction, forming a single phase.

A heterogeneous transition metal-based catalyst is a catalyst that is not soluble, involving a reaction involving two phases, e.g. liquid-solid.

A supported bimetallic catalyst consists of a support, on the surface of which particles of two metals are dispersed and immobilized either in the form of oxides of these metals, or in reduced form, or even a mixture of these two forms.

The support is advantageously an oxide such as alumina or silica.

It is understood that the bimetallic catalyst of two metals, Metal-1 and Metal-2, includes on the support, on its surface accessible to the substrates of the catalysed reaction, species of the two metals, Metal-1 and Metal-2.

A monometallic supported catalyst therefore consists of a support on which particles of a single metal are dispersed and immobilized, either in the form of oxides or in reduced form, or even a mixture of these two forms.

Generally, hydrogenation reaction from oxalate to ethylene glycol is catalysed by a catalyst comprising a metal. For example, the hydrogenation step is carried out with a copper-based catalyst.

In the article by Yuxi Xu et al. (Catal. Sci. Technol, 2022, 12 6782) the use of copper monometallic catalysts supported on zirconium dioxide Cu/ZrO₂ for hydrogenation of oxalates to ethylene glycol is described. The authors studied the effect of the crystalline phases of the substrate synthesized at different temperatures and relate the structure of the catalyst to the catalytic properties.

In the article by Kong et al. (Catalysis Communications, 65, 2015, 46-50) the use of cobalt-based catalysts in the presence of copper as a promoter, supported on zinc oxide CoCu/ZnO for the hydrogenation of oxalates, is described. The monometallic catalyst Co/ZnO for the oxalate hydrogenolysis reaction exhibits selectivity towards methane formation (80%) without ethylene glycol formation. The presence of copper as a promoter makes it possible to direct selectivity towards the formation of ethylene glycol.

For many years, there is a need for a low-cost, environmentally friendly ethylene glycol preparation route.

To date, a heterogeneous catalyst is being sought, easy to prepare, allowing an efficient synthesis of ethylene glycol environmentally clean from oxalate, easily industrialized and safe.

Ethylene glycol is also a model compound for cleavage by the action of hydrogen, namely hydrogenolysis, of the C—C and/or C—O bonds of the diol groups —CHOH—CHOH— present in the carbohydrate molecules (polyols) contained in the biomass. Thus, a catalyst enabling the cleavage of the C—C and C—O bonds of ethylene glycol by hydrogenolysis would enable a depolymerization of the biomass by cleavage of the C—C and/or C—O bonds of the diol groups present in the biomass.

One of the aims of the invention is to propose a method for preparing ethylene glycol from oxalate by a reaction using a heterogeneous bimetallic catalyst, in particular based on copper and cobalt.

Another aim of the invention is a method for preparing ethylene glycol having efficient yields and high selectivity.

Another aim of the invention is the preparation of ethylene glycol that does not use toxic reagents.

Another aim of the invention is the preparation of ethylene glycol, using recyclable or recycled reagents and an efficient and reusable heterogeneous catalyst.

Another aim of the invention is to provide a method for hydrogenolysis of ethylene glycol using a heterogeneous bimetallic catalyst.

Another aim of the invention is to provide a biomass depolymerization method using a heterogeneous bimetallic catalyst.

Another aim of the invention is to provide a new heterogeneous bimetallic catalyst on support. Another aim of the invention is to provide a heterogeneous catalyst usable in a continuous flow method.

Another aim of the present invention is to provide a simple, industrialized and optimized method for preparing this catalyst.

### Use

A first object of the present invention is the use of a supported bimetallic catalyst of formula CoCu/Support, comprising cobalt and copper on a support, in the implementation of a method of preparation of ethylene glycol from an oxalate compound by hydrogenation reaction of said oxalate compound by hydrogen (H₂) to obtain ethylene glycol.

In the present invention the terms “hydrogen” and “dihydrogen” define the same H₂ molecule. It is understood that the CoCu/Support catalyst is a heterogeneous catalyst, the catalyst being in the solid phase and the reactants being in liquid or gaseous form.

The advantage of using a heterogeneous catalyst is that it facilitates the separation of the catalyst from the other species involved in the reaction, allowing the catalyst to be easily recovered and reused and limiting contamination of the product with transition metals.

The use of a heterogeneous catalyst also has the advantage of making it possible to fix the catalyst in the reactor in an enclosure such as a cartridge when operating under continuous flow and thus to obtain products at the reactor outlet free of catalysts.

For the purposes of the present invention, “catalyst” or “supported catalyst” means a material consisting of a support on which catalytic sites are located. It is understood that the bimetallic cobalt and copper catalyst includes a support and atoms of the elements of cobalt and copper.

The total weight of the catalyst corresponds to the weight of the support and that of the cobalt and copper elements.

According to a particular embodiment, the invention relates to the use of a supported bimetallic CoCu/Support catalyst, comprising copper and cobalt on a support, in the implementation of a method of preparation of ethylene glycol from an oxalate compound by hydrogenation reaction of said oxalate compound by hydrogen (H₂) to obtain ethylene glycol, wherein said support is selected from zirconium dioxide (ZrO₂), silica (SiO₂) or alumina (Al₂O₃), in particular zirconium dioxide (ZrO₂).

Use with a CoCu/ZrO₂ Catalyst

According to a particular embodiment, the invention relates to the use of a supported bimetallic catalyst comprising cobalt and copper on a zirconium dioxide support, of the formula CoCu/ZrO₂, in the implementation of a method of preparation of ethylene glycol from an oxalate compound by hydrogenation reaction of said oxalate compound by hydrogen (H₂) to obtain ethylene glycol.

The inventors unexpectedly observed a synergistic effect between copper and cobalt in a heterogeneous supported bimetallic catalyst that significantly increased the yield and selectivity of the oxalate hydrogenation reaction to ethylene glycol. Although the monometallic cobalt-on-zirconium dioxide catalyst is inactive for the oxalate hydrogenation reaction to ethylene glycol, the tests (see examples 21 and 22) showed that cobalt has a promoter function in CuCo/ZrO₂ catalysts promoting yield and selectivity towards ethylene glycol.

Indeed, they found a yield of around 12% with a selectivity of 13% when using a monometallic copper catalyst and that monometallic cobalt-based catalysts are inactive. The use of a bimetallic cobalt and copper catalyst on an oxide support according to the invention makes it possible to achieve a yield of 84% and a selectivity of 95%.

The inventors also observed that a ZrO₂ zirconium dioxide support achieved better results compared to other oxide supports such as silica (SiO₂), cerium dioxide (CeO₂) or alumina (γ-Al₂O₃).

According to a particular embodiment, the invention relates to the use of a supported bimetallic catalyst comprising cobalt and copper on a zirconium dioxide support, of the formula CoCu/ZrO₂, in the implementation of a method of preparation of ethylene glycol from an oxalate compound by hydrogenation reaction of said oxalate compound by hydrogen (H₂) to obtain ethylene glycol.

According to a particular embodiment, the invention relates to the use as defined above, wherein said oxalate is selected from dimethyloxale, diethyloxalate, dibenzyloxalate, diterbutyloxalate, diisopropyloxalate, diphenyloxalate, in particular selected from dimethyloxale or diethyloxalate, preferably diethyloxalate.

Advantageously, the oxalates used can be obtained by a carbonylation reaction of an alcohol in the presence of CO and oxygen.

Advantageously, carbon monoxide CO for the preparation of oxalate is produced by the electrolysis of carbon dioxide CO₂ into carbon monoxide CO.

Thus, the synthesis of ethylene glycol is advantageously carried out from the recovery of CO₂ and an alcohol.

Use with Catalyst Preparation by Soft Chemistry

According to a particular embodiment, the invention relates to the use as defined above, wherein said catalyst is prepared by mixing the support in powder form and an aqueous solution of copper and cobalt salts, followed by drying and calcination.

According to a particular embodiment, the invention relates to the use as defined above, wherein said catalyst is prepared by mixing the support in powder form and an aqueous solution of copper and cobalt salts, devoid of additives or surfactants, in particular without ammonia solution, followed by drying of the obtained material and its calcination.

It is understood that the mixture of the support in powder form and an aqueous solution of copper and cobalt salts consists of bringing a solution of cobalt and copper salts into contact with a support and therefore in an impregnation of the support by the cobalt and copper salts, namely the deposition of cobalt and copper atoms on the surface of the support.

“Drying” means an operation consisting of heating the material in ambient air in a closed enclosure at a temperature of the order of 60 to 100° C. for 10 to 24 hours in order to dry the material, namely to remove the water molecules, namely free water, adsorbed on the surface or interstitial water.

“Calcination” means an operation consisting of heating the solid material in ambient air in a closed enclosure at a high temperature of the order of 400 to 1,000° C. in order to activate it or to modify the physical characteristics of the support and to eliminate the combined water and the salts of metallic precursors such as nitrates and acetates. After calcination, the said material is devoid of water.

Advantageously, the catalyst used is prepared as follows with a preparation method comprising the following steps:
a step A of impregnating a cobalt salt and a copper salt, dissolved in an aqueous solution, in a volume of water from 5 to 10 mL, on a zirconium dioxide support in powder form, with a ratio of solution mass to support mass from 0.6 to 1.0;
to obtain the CoCu/ZrO₂ catalyst in the form of a homogeneous mixture,
a step B of drying said homogeneous mixture, to obtain the CoCu/ZrO₂ catalyst in the form of a dry homogeneous mixture,
an activation step C, comprising calcination of said dry homogeneous mixture to obtain said catalyst.

For the purposes of the present invention, “homogeneous mixture” means the homogeneous material obtained by mixing an aqueous solution of metal salts with the support in the form of a solid powder, said aqueous solution of metal salts being homogeneously distributed on the surface of the particles constituting the support and in the interstices of said particles.

“Dry homogeneous mixture” means a homogeneous mixture in which the majority of free water molecules, adsorbed on the surface or in the interstices of the particles of the support, are removed, for example by drying at 60 to 100° C., in particular 80° C. to obtain a powder. The preparation of the catalyst leads to the formation of the corresponding non-soluble metal oxides because the metal salts of the metals, such as nitrate, are completely eliminated after the calcination step at 600° C.

Advantageously, in step A of impregnation, said aqueous solution containing the cobalt and copper salts is devoid of additives and surfactants. In other words, said aqueous impregnation solution consists of a demineralized solution of water in which the cobalt and copper salts are dissolved.

Advantageously, cobalt salt is cobalt nitrate and copper salt is copper nitrate.

The use according to the invention is therefore carried out with a catalyst prepared with an aqueous solution, a green solvent that is not dangerous compared to the organic solvents used in the prior art. Advantageously, additional additives and surfactants in the synthesis of the catalyst are not necessary, which makes it more industrializable at a lower cost.

A “green solvent” is a non-toxic, biodegradable or agro-based substitute solvent that has the same properties as the toxic solvents it replaces.

Use with a Catalyst Having a Characteristic Relating to the Specific Surface Area.

According to a particular embodiment, the invention relates to the use as defined above, wherein said catalyst has a surface area, analysed by BET, from 1 to 250 m²/g. The range from 1 to 250 m²/g includes the following ranges; from 1 to 25 m²/g; from 25 to 50 m²/g; from 50 to 75 m²/g; from 75 to 100 m²/g; from 100 to 125 m²/g; from 125 to 150 m²/g; from 150 to 175 m²/g; from 175 to 200 m²/g; from 200 to 225 m²/g; from 225 to 250 m²/g. According to a particular embodiment, the invention relates to the use as defined above, wherein said catalyst has a surface area, analysed by BET, from 1 to 50 m²/g.

The range from 1 to 50 m²/g includes the following ranges: from 1 to 10 m²/g; from 10 to 20 m²/g; from 20 to 30 m²/g; from 30 to 40 m²/g; from 40 to 50 m²/g.

According to a particular embodiment, the invention relates to the use as defined above, wherein said catalyst has a surface area, analysed by BET, from 1 to 10 m²/g, preferably about 5 m²/g.

The range from 1 to 10 m²/g includes the following ranges: from 1 to 2 m²/g; from 2 to 3 m²/g; from 3 to 4 m²/g; from 4 to 5 m²/g; from 5 to 6 m²/g; from 6 to 7 m²/g; from 7 to 8 m²/g; from 8 to 9 m²/g, from 9 to 10 m²/g, in particular about 5 m²/g.

The inventors unexpectedly found that a CoCu/ZrO₂ bimetallic catalyst with a low specific surface area of 1 to 50 m²/g, in particular of 1 to 10 m²/g, prepared with a ZrO₂ support with a specific surface area of 5 to 7 m²/g, allowed improved yield and selectivity for the hydrogenation reaction of oxalates into ethylene glycol, compared to a CoCu/ZrO₂ catalyst with the same cobalt and copper content prepared with a ZrO₂ support with a specific surface area greater than 50 m²/g, analysed by BET. Thus, unexpectedly, a catalyst with a low specific surface area of about 5 m²/g is more efficient than a catalyst with a specific surface area greater than 50 m²/g in terms of yield and selectivity for the hydrogenation reaction of oxalates to ethylene glycol. It would rather be expected that a catalyst with a larger specific surface area would be more favourable in terms of catalysis because it favours exchanges.

Use with a Catalyst Having a Specific Crystal Structure

According to a particular embodiment, the invention relates to the use as defined above, wherein said catalyst comprises a crystalline phase, analysed by X-ray diffraction, crystallized in a monoclinic crystalline system.

A “monoclinic crystal system” refers to a system whose point group of symmetry is one of the following point groups: 2, m or 2/m.

According to a particular embodiment, the invention relates to the use as defined above, wherein said crystalline phase represents from 50 to 90% by total weight of the catalyst.

According to a particular embodiment, the invention relates to the use as defined above, wherein said crystalline phase is baddeleyite.

According to a particular embodiment, the invention relates to the use as defined above, wherein the catalyst support is natural zirconium dioxide (ZrO₂) crystallizing in monoclinic baddeleyite.

For the purposes of the present invention, “baddeleyite” means a natural zirconium oxide of the formula ZrO₂, containing from 0.1% to 5% hafnium oxide, and crystallizing in a monoclinic crystalline system. The characteristics of baddeleyite, such as the composition and crystallographic structure, in particular the space group with the dimensions of the crystalline lattice, are reported and accessible in the prior art and known to the skilled person in the art, such as in Kudoh, Y. et al., (Phys Chem Minerals 13, 233-237 (1986)) or Mccullough J D. et al. (Acta Crystallographica 12 (1959) 507-511).

According to a particular embodiment, the invention relates to the use as defined above, wherein the catalyst support made of zirconium dioxide ZrO₂ comprises impurities such as Hafnium (Hf), Rhenium (Re) and Silicon (Si) atoms.

Advantageously, the percentage mass ratio between Hafnium atoms and zirconium atoms (Hf/Zr) is less than 5%.

Advantageously, the percentage mass ratio between Rhenium atoms and zirconium atoms (Re/Zr) is less than 5%.

Advantageously, the percentage mass ratio between silicon atoms and zirconium atoms (Si/Zr) is less than 2%.

According to a particular embodiment, the invention relates to the use as defined above, wherein said catalyst has a microstructure whose crystallite size is from 15 to 100 nm, preferably from 15 to 50 nm.

The crystal structure of the catalyst can be analysed by powder X-ray diffraction. The analysis of the phases present and that of their structure is carried out by assigning the diffraction peaks present in the diffraction diagram obtained in comparison with reference files.

The crystalline phase of the baddeleyite of zirconium dioxide ZrO₂ is for example indicated in the ICDD reference file n° 00-037-1484.

From the diffraction pattern, the size of the crystallites can be evaluated according to the following Scherrer formula:

$$t = \frac{k \cdot λ}{H \cdot cos θ} \begin{matrix}t = crystallite size \\ k = correction factor = 0 , 89 \\ λ = source wavelength \\ H = length at half height of peak \\ θ := diffraction angle\end{matrix}$$

The range “from 15 to 100 nanometers” includes the following ranges: from 15 to 20 nm; from 20 to 30 nm; from 30 to 40 nm; from 40 to 50 nm; from 50 to 60 nm; from 60 to 70 nm; from 70 to 80 nm; from 80 to 90 nm; from 90 to 100 nm.

According to a particular embodiment, the invention relates to the use as defined above, wherein:
said catalyst has a surface area, analysed by BET, from 1 to 250 m²/g, in particular from 1 to 50 m²/g,
and said catalyst comprises a crystalline phase, analysed by X-ray diffraction, crystallized in a monoclinic crystalline system.

According to a particular embodiment, the invention relates to the use as defined above, wherein:
said catalyst has a surface area, analysed by BET, from 1 to 10 m²/g,
and said catalyst comprises a crystalline phase, analysed by X-ray diffraction, crystallised in monoclinic baddeleyite, the size of which is preferably from 15 to 100 nm, and
optionally including impurities such as Hafnium (Hf), Rhenium (Re) and Silicon (Si) atoms.

According to a particular embodiment, the invention relates to the use as defined above, in which said catalyst has a surface area, analysed by BET, from 1 to 250 m²/g, in particular from 1 to 50 m²/g, preferably from 1 to 10 m²/g, notably about 5 m²/g,
optionally in which said catalyst comprises a crystalline phase, analysed by X-ray diffraction, crystallized in a monoclinic crystalline system, in particular said crystalline phase represents from 50 to 90% by total weight of the catalyst, preferably said crystalline phase is baddeleyite and the crystallite size of which is preferably from 15 to 100 nm.
   Use with a Catalyst Having Specific Cu and Co Contents

According to a particular embodiment, the invention relates to the use as defined above, wherein the total content of the bimetallic elements copper and cobalt is from 0.5 to 25% by total weight of the catalyst.

The total weight of the catalyst includes the mass of the support and the mass of the cobalt and copper bimetallic elements.

The range from 0.5 to 25% includes the following ranges: from 0.5 to 5%; from 5 to 10%; from to 15%; from 15 to 20%; from 20 to 25%.

According to a particular embodiment, the invention relates to the use as defined above, wherein the total content of the bimetallic elements copper and cobalt is from 5 to 20% by total weight of the catalyst.

“Total content of the bimetallic elements copper and cobalt” means a content corresponding to the addition of the elements copper and cobalt in the catalyst.

According to a particular embodiment, the invention relates to the use as defined above, wherein the total content of the element copper is from 1 to 25% by total weight of the catalyst, in particular from 1 to 10% by total weight of the catalyst.

According to a particular embodiment, the invention relates to the use as defined above, wherein the total content of the element cobalt is from 1 to 25% by total weight of the catalyst, in particular from 1 to 10% by total weight of the catalyst.

According to a particular embodiment, the invention relates to the use as defined above, wherein:
the total content of the element copper is from 1 to 25% by total weight of the catalyst, in particular from 1 to 10% by total weight of the catalyst,
and the total cobalt element content is from 1 to 25% by total weight of the catalyst, in particular from 1 to 10% by total weight of the catalyst.

According to a particular embodiment, the invention relates to the use as defined above, wherein the mass quantity of copper is greater than that of cobalt.

According to a particular embodiment, the invention relates to the use as defined above, wherein the mass quantity of cobalt is greater than that of copper.

According to a particular embodiment, the invention relates to the use as defined above, wherein the ratio by weight between copper and cobalt varies from 1:1 to 1:10, in particular from 1:1 to 1:5.

According to a particular embodiment, the invention relates to the use as defined above, wherein the mass composition of cobalt and copper of the catalyst is selected from Co(10%)Cu(5%) and Co(15%)Cu(5%).

According to a particular embodiment, the invention relates to the use as defined above, wherein the ratio by weight between cobalt and copper varies from 1:1 to 1:10.

According to a particular embodiment, the invention relates to the use as defined above, wherein the ratio by weight between cobalt and copper varies from 1:1 to 1:5.

According to a particular embodiment, the invention relates to the use as defined above, wherein:
the ratio by weight between cobalt and copper varies from 1:1 to 1:5,
the copper content is 10% by total weight of the catalyst
and the cobalt content is greater than or equal to 2% by total weight of the catalyst.

According to a particular embodiment, the invention relates to the use as defined above, wherein
the ratio by weight between cobalt and copper ranges from 1:2 to 1:5,
the copper content is 10% by total weight of the catalyst,
and the cobalt content is greater than or equal to 2% by total weight of the catalyst.

According to a particular embodiment, the invention relates to the use as defined above, wherein:
the weight ratio between cobalt and copper varies from 1:2 to 1:5,
the copper content is 10% by total weight of the catalyst,
the cobalt content is greater than or equal to 2% by total weight of the catalyst
and the specific surface area measured by BET is from 1 to 10 m²/g.

The inventors surprisingly observed that when the CoCu/ZrO₂ catalyst comprises a mass quantity of copper greater than the mass quantity of cobalt, in particular with a ratio by weight between cobalt and copper varying from 1:1 to 1:5 and when the copper content is 10% by total weight of the catalyst and when the cobalt content is greater than or equal to 2% by weight, the yield of the hydrogenation reaction and its selectivity are improved, with values respectively greater than 50% in yield and greater than 70% in selectivity.

When the CoCu/ZrO₂ catalyst comprises a mass quantity of copper greater than the mass quantity of cobalt, in particular with a ratio by weight between cobalt and copper varying from 1:2 to 1:5 and when the copper content is 10% by total weight of the catalyst and the cobalt content is greater than or equal to 2% by total weight of the catalyst and the specific surface area of the catalyst is from 1 to 10 m²/g, the yield of the hydrogenation reaction and its selectivity are improved, with values respectively greater than 70% in yield and greater than 80% in selectivity.

According to a particular embodiment, the invention relates to the use as defined above, wherein the mass composition of cobalt and copper of the catalyst is selected from:
Co(2%)Cu(10%);
Co(5%)Cu(10%),
Co(5%)Cu(5%),
and Co(10%)Cu(10%),
   preferably Co(2%)Cu(10%) and Co(5%)Cu(10%),
   more preferentially Co(5%)Cu(10%).

The mass composition Co(5%)Cu(10%) corresponds to a content of 5% Co by total weight of the catalyst and of 10% of Cu by total weight of the catalyst, i.e. a mass ratio between cobalt and copper of 1:2.

The mass composition Co(5%)Cu(5%) corresponds to a content of 5% by weight of Co and 5% by weight of Cu, i.e. a ratio of cobalt to copper of 1:1 with a content of 5% Co by total weight of the catalyst and a content of 5% Cu by total weight of the catalyst.

According to a particular embodiment, the invention relates to the use as defined above, wherein the mass composition of cobalt and copper of the catalyst is selected from Co(2%)Cu(10%); Co(5%)Cu(10%), more preferentially Co(5%)Cu(10%), in which the mass content of copper is greater than the mass content of cobalt.

According to a particular embodiment, the invention relates to the use as defined above, wherein the mass composition of cobalt and copper of the catalyst is Co(5%)Cu(10%) and the specific surface area is from 1 to 10 m²/g.

According to a particular embodiment, the invention relates to the use as defined above, wherein the mass composition of cobalt and copper of the catalyst varies from Co(2%)Cu(10%) to Co(10%)Cu(10%).

According to a particular embodiment, the invention relates to the use as defined above, wherein:
the total content of the element copper is from 5 to 10% by total weight of the catalyst, in particular 10% by total weight of the catalyst,
and the total content of the element cobalt is from 2 to 10% by total weight of the catalyst, in particular 5% by total weight of the catalyst.

According to a particular embodiment, the invention relates to the use as defined above, wherein the mass composition of cobalt and copper of the catalyst varies from Co(2%)Cu(10%) to Co(5%)Cu(10%).

According to a particular embodiment, the invention relates to the use as defined above, wherein:
the total content of the copper element is 10% by total weight of the catalyst,
and the total content of the cobalt element is from 2 to 5% by total weight of the catalyst, in particular 5% by total weight of the catalyst.

According to a particular embodiment, the invention relates to the use as defined above, wherein:
the total content of the copper element is 10% by total weight of the catalyst,
the total content of the cobalt element is from 2 to 5% by total weight of the catalyst, in particular 5% by total weight of the catalyst,
and the specific surface area of the catalyst is from 1 to 50 m²/g, preferably from 1 to 10 m²/g, analysed by BET.
   Use with a Catalyst Having a Surface Characteristic on Cu and Co

According to a particular embodiment, the invention relates to the use as defined above, wherein said catalyst has a molar amount of more than 50% of the element copper at oxidation degree (II) and/or of the element cobalt at oxidation degree (II).

The inventors unexpectedly found that for the hydrogenation reaction of oxalates into ethylene glycol, the CoCu/ZrO₂ bimetallic catalysts could be used directly after preparation by calcination in air without any prior step of reduction under hydrogen of the copper and cobalt atoms before use of the catalyst in the hydrogenation reaction.

Advantageously, in some cases the use of a catalyst in which a majority of the copper and cobalt elements are already present in the oxidized state may allow the use of the said catalyst without prior reduction of the copper and cobalt elements of the catalyst before the implementation of the hydrogenation reaction of oxalate into ethylene glycol. This also makes it possible to avoid the storage of catalysts in an inert atmosphere in order to avoid the oxidation of copper and cobalt atoms.

According to a particular embodiment, the invention relates to the use as defined above, wherein said catalyst has a molar amount of more than 50% of the element copper at oxidation degree (II) and more than 50% of the element cobalt at oxidation degree (II).

According to a particular embodiment, the invention relates to the use as defined above, wherein said catalyst has a molar amount of more than 70% of the element copper at oxidation degree (II) and more than 65% of the element cobalt at oxidation degree (II).

According to a particular embodiment, the invention relates to the use as defined above, wherein said catalyst has a molar amount of more than 50% of the element copper at oxidation degree (II) in the form of CuO and more than 50% of the element cobalt at oxidation degree (II) in the form of Co₂O₃ or Co(OH)₂.

According to a particular embodiment, the invention relates to the use as defined above, wherein said catalyst has a molar amount greater than 70% of the element copper at oxidation degree (II) in the form of CuO and a molar amount greater than 65% of the element cobalt at oxidation degree (II) in the form of Co₂O₃ or Co(OH)₂.

According to a particular embodiment, the invention relates to the use as defined above, wherein said catalyst has:
a molar amount from 10 to 30% of the element copper in the metallic state at oxidation degree (0) or in oxidation degree (I) in the form of Cu₂O
and a molar amount from 10 to 35% of the element cobalt in the metallic state at oxidation degree (0).

According to a particular embodiment, the invention relates to the use as defined above, wherein said catalyst has:
a molar amount greater than 70% of the element copper at oxidation degree (II) in the form of CuO,
a molar amount from 10 to 30% of the element copper in the metallic state at oxidation degree (0) or at oxidation degree (I) in the form of Cu₂O
a molar amount from 10 to 35% of the element cobalt in the metallic state at oxidation degree (0),
and a molar amount greater than 65% of the element cobalt at oxidation degree oxidation (II) in the form of Co₂O₃ or Co(OH)₂.
   Use with a Catalyst Having a Particular Morphology

According to a particular embodiment, the invention relates to the use as defined above, wherein said catalyst is in the form of a population of micrometric particles from 1 to 500 μm. The range from 1 to 500 μm includes the following ranges: from 1 to 50 μm; from 50 to 100 μm; from 100 to 200 μm; from 200 to 300 μm, from 300 to 400 μm. “Particles” means distinct clusters having either visual or mechanical coherence.

According to a particular embodiment, the invention relates to the use as defined above, wherein said catalyst is in the form of a population of particles of rounded morphology.

For the purposes of the present invention, “rounded particles” means particles that do not have edges, angles or beveled edges.

Use with Hydrogenation Reaction Selectivity

The selectivity of a chemical reaction specifies the amount of the desired product formed in relation to the number of moles consumed of the limiting reactant. It indicates whether several reactions occur in parallel, leading to unwanted by-products, or whether the reaction carried out is the only one that consumes the reactant.

In the case of the hydrogenation step, selectivity is defined as the amount of ethylene glycol obtained in relation to the total quantity of products that have been obtained including ethylene glycol and secondary by-products, resulting from the transformation of the oxalate compound. The term “selective reaction” refers to a reaction that produces the target product, ethylene glycol, with a selectivity of more than 50%.

According to a particular embodiment, the invention relates to the use as defined above, wherein the hydrogenation reaction of said oxalate compound by hydrogen to obtain ethylene glycol is selective, with a selectivity of more than 70%.

According to a particular embodiment, the invention relates to the use as defined above, wherein the hydrogenation reaction of said oxalate compound by hydrogen to obtain ethylene glycol is selective, with a selectivity of more than 80%.

According to a particular embodiment, the invention relates to the use as defined above, wherein the hydrogenation reaction of said oxalate compound by hydrogen to obtain ethylene glycol is selective, with a selectivity of more than 90%.

According to a particular embodiment, the invention relates to the use as defined above, wherein the hydrogenation reaction of said oxalate compound by hydrogen to obtain ethylene glycol is selective, with a selectivity greater than or equal to 95%.

Use with Hydrogenation Reaction Yield

The chemical yield reflects the efficiency of the chemical reaction being studied. The yield refers to the ratio of the amount of product obtained to the maximum amount that would be obtained if the reaction were complete.

The yield of the hydrogenation reaction of oxalate into ethylene glycol according to the invention is determined as a percentage of moles of ethylene glycol obtained per mole of oxalate.

The yield of the hydrogenation reaction of oxalate into ethylene glycol can be determined, in particular, using gas chromatography coupled with a mass spectrometer (GC-MS) in which mesitylene is used as an internal standard.

According to a particular embodiment, the invention relates to the use as defined above, wherein the hydrogenation reaction of said oxalate compound by hydrogen to obtain ethylene glycol has a yield greater than 50%, in particular greater than 70%, preferably greater than 80%.

According to a particular embodiment, the invention relates to the use as defined above, wherein the hydrogenation reaction of said oxalate compound by hydrogen to obtain ethylene glycol is selective, with a selectivity greater than 70%, preferably greater than 90%, more preferably greater than or equal to 95%
and/or wherein the hydrogenation reaction of said oxalate compound by hydrogen to obtain ethylene glycol has a yield greater than 70%, preferably greater than 80%.

### Catalyst

A second object of the invention relates to a supported bimetallic catalyst comprising copper and cobalt on a zirconium dioxide support, of formula CoCu/ZrO₂,
in which said catalyst has a surface area, analysed by BET, from 1 to 250 m²/g.

According to a particular embodiment, the invention relates to a supported bimetallic catalyst as defined above, wherein said catalyst has a surface area, analysed by BET, from 1 to 75 m²/g, preferably from 1 to 50 m²/g.

According to a particular embodiment, the invention relates to a supported bimetallic catalyst as defined above, in which said catalyst has a surface area, analysed by BET, from 1 to 250 m²/g,
and wherein said catalyst comprises a crystalline phase, analysed by X-ray diffraction, crystallized in a monoclinic crystalline system.

### Specific Surface

According to a particular embodiment, the invention relates to a supported bimetallic catalyst as defined above, wherein said catalyst has a surface area, analysed by BET, from 1 to 50 m²/g.

According to a particular embodiment, the invention relates to a supported bimetallic catalyst as defined above, wherein said catalyst has a surface area, analysed by BET, from 1 to 10 m²/g, preferably about 5 m²/g.

### Structure

According to a particular embodiment, the invention relates to a supported bimetallic catalyst as defined above, wherein said crystalline phase represents from 50 to 90% by total weight of the catalyst.

According to a particular embodiment, the invention relates to a supported bimetallic catalyst as defined above, wherein said crystalline phase is Baddeleyite.

According to a particular embodiment, the invention relates to a bimetallic catalyst as defined above, wherein said zirconium dioxide support comprises a crystalline phase, analysed by X-ray diffraction, crystallized in a monoclinic crystalline system.

According to a particular embodiment, the invention relates to a bimetallic catalyst as defined above, said crystalline phase represents from 50 to 90% by total weight of the catalyst. The range from 50 to 90% includes the following ranges: from 50 to 55%, from 55 to 60%, from 60 to 65%, from 65 to 70%, from 70 to 75%, from 75 to 80%, from 80 to 85%, from 85 to 90%.

According to a particular embodiment, the invention relates to a bimetallic catalyst as defined above, wherein said crystalline phase is baddeleyite.

Advantageously, the catalyst according to the invention comprises from 50 to 90% baddeleyite. Advantageously, the support of the zirconium dioxide catalyst according to the invention consists of more than 50% baddeleyite, in particular from 50 to 100% baddeleyite.

According to a particular embodiment, the invention relates to a bimetallic catalyst as defined above, wherein said catalyst comprises a crystalline phase, analysed by X-ray diffraction, comprising a crystallite size from 15 to 100 nm.

According to a particular embodiment, the invention relates to a supported bimetallic catalyst as defined above, wherein the catalyst support made of zirconium dioxide ZrO₂ comprises impurities such as Hafnium (Hf), Rhenium (Re) and Silicon (Si) atoms.

Advantageously, the percentage mass ratio between Hafnium atoms and zirconium atoms (Hf/Zr) is less than 5%.

Advantageously, the percentage mass ratio between Rhenium atoms and zirconium atoms (Re/Zr) is less than 5%.

Advantageously, the percentage mass ratio between silicon atoms and zirconium atoms (Si/Zr) is less than 2%.

According to a particular embodiment, the invention relates to a supported bimetallic catalyst as defined above, wherein said catalyst has a microstructure whose crystallite size is from 15 to 100 nm, preferably from 15 to 50 nm.

According to a particular embodiment, the invention relates to a supported bimetallic catalyst as defined above, wherein:
said catalyst has a surface area, analysed by BET, from 1 to 250 m²/g, in particular from 1 to 50 m²/g, preferably from 1 to 10 m² g,
and said catalyst comprises a crystalline phase, analysed by X-ray diffraction, crystallized in a monoclinic crystalline system.

According to a particular embodiment, the invention relates to a supported bimetallic catalyst as defined above, wherein:
said catalyst has a surface area, analysed by BET, from 1 to 10 m²/g,
and said catalyst comprises a crystalline phase, analysed by X-ray diffraction, crystallised in monoclinic baddeleyite with a crystallite size from 15 to 100 nm, preferably from 15 to 50 nm
and including impurities such as Hafnium (Hf), Rhenium (Re) and Silicon (Si) atoms.

According to a particular embodiment, the invention relates to a supported bimetallic catalyst as defined above, comprising copper and cobalt on a zirconium dioxide support, of the formula CoCu/ZrO₂, in which said catalyst has a surface area, analysed by BET, from 1 to 250 m²/g, preferably from 1 to 50 m²/g, preferably from 1 to 10 m²/g, in particular from about 5 m²/g and wherein said catalyst comprises a crystalline phase, analysed by X-ray diffraction, crystallized in a monoclinic crystalline system, in particular said crystalline phase represents 50 to 90% by total weight of the catalyst, preferably said crystalline phase is baddeleyite and preferably comprising a crystallite size from 15 to 100 nm.

### Composition

According to a particular embodiment, the invention relates to a supported bimetallic catalyst as defined above, wherein said catalyst comprises a total content of the bimetallic elements copper and cobalt from 0.5 to 25% by total weight of the catalyst.

According to a particular embodiment, the invention relates to a supported bimetallic catalyst as defined above, wherein said catalyst comprises a total content of the bimetallic elements copper and cobalt from 5 to 20%.

According to a particular embodiment, the invention relates to a supported bimetallic catalyst as defined above, wherein the total content of the element copper is from 1 to 25% by total weight of the catalyst, preferably from 1 to 10% by total weight of the catalyst.

According to a particular embodiment, the invention relates to a supported bimetallic catalyst as defined above, wherein the total content of the element cobalt is from 1 to 25% by total weight of the catalyst, preferably from 1 to 10% by total weight of the catalyst.

According to a particular embodiment, the invention relates to a catalyst as defined above, wherein the mass quantity of cobalt is greater than that of copper.

According to a particular embodiment, the invention relates to a catalyst as defined above, wherein the ratio by weight between copper and cobalt varies from 1:1 to 1:10, in particular from 1:1 to 1:5.

According to a particular embodiment, the invention relates to a catalyst as defined above, wherein the mass composition of cobalt and copper of the catalyst is selected from Co(10%)Cu(5%) and Co(15%)Cu(5%).

In a particular embodiment, the invention relates to a catalyst as defined above, wherein the mass quantity of copper is greater than that of cobalt.

According to a particular embodiment, the invention relates to a supported bimetallic catalyst as defined above, wherein the ratio by weight between cobalt and copper varies from 1:1 to 1:10.

According to a particular embodiment, the invention relates to a supported bimetallic catalyst as defined above, wherein the ratio by weight between cobalt and copper varies from 1:1 to 1:5.

According to a particular embodiment, the invention relates to a supported bimetallic catalyst as defined above, wherein the ratio by weight between cobalt and copper varies from 2:10 to 5:10.

According to a particular embodiment, the invention relates to a supported bimetallic catalyst as defined above, wherein the mass composition of cobalt and copper of the catalyst is selected from:
Co(2%)Cu(10%);
Co(5%)Cu(10%),
Co(5%)Cu(5%),
and Co(10%)Cu(10%),
   preferably Co(2%)Cu(10%) and Co(5%)Cu(10%),
   more preferentially Co(5%)Cu(10%).

According to a particular embodiment, the invention relates to a supported bimetallic catalyst as defined above, wherein the mass composition of cobalt and copper of the catalyst is selected from Co(2%)Cu(10%) and Co(5%)Cu(10%), more preferentially Co(5%)Cu(10%), in which the mass content of copper mass content is greater than the mass content of cobalt. According to a particular embodiment, the invention relates to a supported bimetallic catalyst as defined above, wherein the mass composition of cobalt and copper of the catalyst is Co(5%)Cu(10%) and the specific surface area is from 1 to 10 m²/g.

According to a particular embodiment, the invention relates to a supported bimetallic catalyst as defined above, wherein the mass composition of cobalt and copper of the catalyst varies from Co(2%)Cu(10%) to Co(10%)Cu(10%).

According to a particular embodiment, the invention relates to a supported bimetallic catalyst as defined above, wherein
the total content of the element copper is from 5 to 10% by total weight of the catalyst, in particular 10% by total weight of the catalyst,
and the total content of the element cobalt is 2 to 10% by total weight of the catalyst, in particular 5% by total weight of the catalyst.

According to a particular embodiment, the invention relates to a supported bimetallic catalyst as defined above, wherein the mass composition of cobalt and copper of the catalyst varies from Co(2%)Cu(10%) to Co(5%)Cu(10%).

According to a particular embodiment, the invention relates to a supported bimetallic catalyst as defined above, wherein:
the total content of the copper element is 10% by total weight of the catalyst,
and the total content of cobalt element is from 2 to 5% by total weight of the catalyst, in particular 5% by total weight of the catalyst.

According to a particular embodiment, the invention relates to a supported bimetallic catalyst as defined above, wherein:
the total content of cobalt element is from 2 to 5% by total weight of the catalyst, in particular 5% by total weight of the catalyst,
and the specific surface area of the catalyst is from 1 to 50 m²/g, preferably from 1 to 10 m²/g, analysed by BET.

According to a particular embodiment, the invention relates to a supported bimetallic catalyst as defined above, wherein:
the total content of cobalt element is from 2 to 5% by total weight of the catalyst, in particular 5% by total weight of the catalyst,
the specific surface area of the catalyst is from 1 to 50 m²/g, preferably from 1 to 10 m²/g, analysed by BET,
and comprises a crystalline phase, analysed by X-ray diffraction, crystallised in a monoclinic crystalline system, in particular baddeleyite, the size of which is preferably from 15 to 100 nm, preferably from 15 to 50 nm and in particular including Hafnium, Rhenium and Silicon impurities.

### Degree of Oxidation of Co and Cu at the Surface

According to a particular embodiment, the invention relates to a supported bimetallic catalyst as defined above, wherein said catalyst has a molar amount of more than 50% of the element copper at oxidation degree (II) and/or of the element cobalt at oxidation degree (II).

Advantageously, a bimetallic catalyst in which the majority of the elements copper and cobalt are already present in the oxidized state allows said catalyst to be used without prior reduction of the copper and cobalt elements of the catalyst before the implementation of the hydrogenation reaction of oxalate into ethylene glycol. This also makes it possible to avoid the storage of catalysts in an inert atmosphere in order to avoid the oxidation of copper and cobalt atoms.

According to a particular embodiment, the invention relates to a catalyst as defined above, wherein said catalyst has a molar amount of more than 50% of the element copper at oxidation degree (II) and more than 50% of the element cobalt at oxidation degree (II).

According to a particular embodiment, the invention relates to a catalyst as defined above, wherein said catalyst has a molar amount of more than 70% of the element copper at oxidation degree (II) and more than 65% of the element cobalt at oxidation degree (II).

According to a particular embodiment, the invention relates to a catalyst as defined above, wherein said catalyst possesses a molar amount of more than 50% of the element copper at oxidation degree (II) in the form of CuO and more than 50% of the element cobalt at oxidation degree (II) in the form of Co₂O₃ or Co(OH)₂.

According to a particular embodiment, the invention relates to a catalyst as defined above, wherein said catalyst has a molar amount greater than 70% of the element copper at oxidation degree (II) in the form of CuO and a molar amount greater than 65% of the element cobalt at oxidation degree (II) in the form of Co₂O₃ or Co(OH)₂.

According to a particular embodiment, the invention relates to a catalyst as defined above, wherein said catalyst has:
a molar amount from 10 to 30% of the element copper in the metallic state at oxidation degree (0) or in oxidation degree (I) in the form of Cu₂O
and a molar amount from 10 to 35% of the element cobalt in the metallic state at oxidation degree (0).

According to a particular embodiment, the invention relates to a catalyst as defined above, wherein said catalyst has:
a molar amount greater than 70% of the element copper at oxidation degree (II) in the form of CuO,
a molar amount from 10 to 30% of the element copper in the metallic state at oxidation degree (0) or at oxidation degree (I) in the form of Cu₂O
a molar amount from 10 to 35% of the element cobalt in the metallic state at oxidation degree (0),
and a molar amount greater than 65% of the element cobalt at oxidation degree oxidation (II) in the form of Co₂O₃ or Co(OH)₂.

According to a particular embodiment, the invention relates to a catalyst as defined above, wherein said catalyst has:
a molar amount of more than 50% of the element copper at oxidation degree (II) and more than 50% of the element cobalt at oxidation degree (II),
a specific surface area from 1 to 50 m²/g
a crystalline phase, analyzed by X-ray diffraction, crystallized in a monoclinic crystal system. According to a particular embodiment, the invention relates to a catalyst as defined above, wherein said catalyst has:
   a molar amount greater than 70% of the element copper at oxidation degree (II) in the form of CuO,
   a molar amount from 10 to 30% of the element copper in the metallic state at oxidation degree (0) or at oxidation degree (I) in the form of Cu₂O
   a molar amount from 10 to 35% of the element cobalt in the metallic state at oxidation degree (0),
   an molar amount greater than 65% of the element cobalt at oxidation degree (II) in the form of Co₂O₃ or Co(OH)₂
   a specific surface area from 1 to 50 m²/g, in particular from 1 to 10 m²/g analysed by BET,
   and a crystalline phase, analysed by X-ray diffraction, crystallized in a monoclinic crystal system, notably baddeleyite, in particular comprising Hafnium, Rhenium and Silicon impurities.

Advantageously in said catalyst as defined:
the percentage mass ratio between Hafnium atoms and zirconium atoms (Hf/Zr) is less than 5%,
the percentage mass ratio between Rhenium atoms and zirconium atoms (Re/Zr) is less than 5%,
and the percentage mass ratio between silicon atoms and zirconium atoms (Si/Zr) is less than 2%.

### Morphology

According to a particular embodiment, the invention relates to a supported bimetallic catalyst as defined above, wherein said catalyst is in the form of micrometric particles from 1 to 500 μm.

These particles are in particular made up of agglomerate of rods with a ratio between length and thickness from 1 to 10, and an average thickness from 50 to 500 nm.

As a non-limiting example, morphology and average size can be assessed by scanning electron microscopy (SEM).

Micrometric sizes allow easier handling of the catalyst.

According to a particular embodiment, the invention relates to a supported bimetallic catalyst as defined above, wherein said catalyst comprises a total content of the bimetallic elements copper and cobalt from 0.5 to 25% by total weight of the catalyst, preferably the ratio by weight between cobalt and copper varies from 1:1 to 1:10, preferably from 1:1 to 1:5, in particular, the mass composition of cobalt and copper of the catalyst is selected from:
Co(2%)Cu(10%),
Co(5%)Cu(10%),
Co(5%)Cu(5%),
and Co(10%)Cu(10%),
   preferably Co(2%)Cu(10%) and Co(5%)Cu(10%),
   more preferentially Co(5%)Cu(10%),
   and/or wherein said catalyst has a molar amount of more than 50% of the element copper at oxidation degree (II) and/or of the element cobalt at oxidation degree (II),
   and/or wherein said catalyst is in the form of a population of micrometric particles from 1 to 500 μm, preferably in the form of a population of particles of rounded morphology.

### Characteristics of the Catalyst in a Reducing Atmosphere

According to a particular embodiment, the invention relates to a supported bimetallic catalyst as defined above, wherein said catalyst analysed by temperature-programmed reduction (TPR) carried out in a dihydrogen reducing atmosphere over a temperature range from 30 to 900° C., is characterized by a reduction temperature of metallic copper of oxidation degree (0) in the range from 150 to 250° C.

Advantageously said reduction temperature of metallic copper is in a temperature range from 170 to 180° C., preferably 174° C.

Advantageously, at a temperature higher than said reduction temperature of metallic copper, in particular by at least 10° C., copper atoms are more than 80%, in particular from 80 to 100%, preferably 100% in metallic form.

The range from 80 to 100% includes the following ranges: from 80 to 85%, from 85 to 90%, from 90 to 95%, from 95 to 96%, from 96 to 97%, from 97 to 98%, from 98 to 99%, from 99 to 100%.

Advantageously, at a temperature higher than said reduction temperature of metallic copper, in particular by at least 10° C., the cobalt atoms are 80 to 100%, preferably 100% in oxidized state, i.e. at an oxidation degree greater than zero.

Advantageously, at a temperature higher than said reduction temperature of metallic copper, in particular by at least 10° C., cobalt atoms are from 0 to 20%, in particular from 0 to 10%, preferably less than 5%, in metallic state at oxidation degree zero.

The range from 0 to 20% includes the following values: 0.0%; 0.05%, 1%, 2%, 3%, 4%, 5%, 6%, 7%, 8%, 9%, 10%, 11%, 12%, 13%, 14%, 15%, 16%, 17%, 18%, 19%, 20%.

According to a particular embodiment, the invention relates to a supported bimetallic catalyst as defined above, wherein said catalyst analysed by temperature-programmed reduction (TPR) carried out in a dihydrogen reducing atmosphere over a temperature range from 30 to 900° C., is characterized by a reduction temperature of metallic copper of oxidation degree (0) in the range from 150 to 250° C.,
in particular at a temperature higher by at least 10° C. than said reduction temperature of metallic copper analysed by TPR,
copper atoms are from 80 to 100%, preferably 100% in metallic form,
cobalt atoms are from 80 to 100%, preferably 100% in oxidized form.

According to a particular embodiment, the invention relates to a supported bimetallic catalyst as defined above, wherein said catalyst analysed by temperature-programmed reduction (TPR) carried out in a reducing atmosphere under a flux of 5 vol. % H₂ in Argon, in particular at 30 mL/min, over a temperature range from 30 to 900° C., in particular with a heating rate of 5° C./min, is characterized by a reduction temperature of metallic copper of oxidation degree (0) in the range from 150 to 250° C.

Advantageously, the catalyst is pre-treated in an inert atmosphere before the said analysis, in particular at 200° C. under Helium.

According to a particular embodiment, the invention relates to a supported bimetallic catalyst as defined above, wherein said catalyst in reducing atmosphere comprising hydrogen at a pressure from 2 to 10 MPa and at a temperature from 150 to 250° C., in particular from 180 to 220° C., comprises:
from 80 to 100%, in particular from 95 to 100%, preferably 100%, of metallic copper atoms Cu(0) and
from 80 to 100% of cobalt atoms at an oxidation degree greater than zero.

Advantageously copper atoms are 95%, preferably 100% in metallic form.

Advantageously cobalt atoms are from 80 to 100%, in particular from 95 to 100%, preferably 100%, in oxidized form at an oxidation degree greater than zero.

Advantageously cobalt atoms are from 0 to 20%, in particular from 0 to 15%, preferably less than 20%, in metallic form.

### Ethylene Glycol Preparation Method

A third object of the invention relates to a method of preparation of ethylene glycol comprising:
a hydrogenation step by hydrogen of an oxalate compound to ethylene glycol, in the presence of a supported bimetallic catalyst comprising copper and cobalt on a zirconium dioxide support, of formula CoCu/ZrO₂.

According to a particular embodiment, the invention relates to a method of preparation as defined above, wherein said oxalate compound is of the following Formula 1:

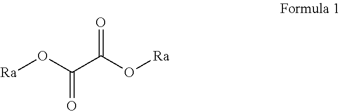

in which Rₐ represents:
a C₁ to C₂₀ linear or branched alkyl group,
a C₃ to C₁₀ cycloalkyl group,
a C₃ to C₂₀ aryl or heteroaryl group,
a C₅ to C₂₀ alkyl-aryl or alkyl-heretoaryl group.

According to a particular embodiment, the invention relates to a method of preparation of ethylene glycol as defined above wherein said oxalate compound is selected from dimethyloxalate, diethyloxalate, diphenyloxalate, dibenzyloxalate, isopropyloxalate and diterbutyloxalate.

According to a particular embodiment, the invention relates to a method of preparation of ethylene glycol as defined above, wherein:
the hydrogenation step comprises:
   bringing into contact:
      said oxalate compound,
      dihydrogen,
      said supported catalyst of formula CoCu/ZrO₂, comprising cobalt and copper on zirconium dioxide support,
      optionally a solvent,
   to obtain a reaction medium that may be pressurized,
   optionally heating said reaction medium,
      to obtain ethylene glycol.

According to a particular embodiment, the invention relates to a method of preparation of ethylene glycol as defined above, wherein:
the hydrogenation step comprises
   bringing into contact:
      said oxalate compound,
      dihydrogen,
      said supported catalyst of formula CoCu/ZrO₂, comprising cobalt and copper on zirconium dioxide support,
      a solvent,
   to obtain a reaction medium,
   optionally heating said reaction medium,
      to obtain ethylene glycol.

According to a particular embodiment, the invention relates to a method of preparation of ethylene glycol as defined above, wherein:
the hydrogenation step comprises
   bringing into contact:
      said oxalate compound,
      dihydrogen,
      said supported catalyst of formula CoCu/ZrO₂, comprising cobalt and copper on zirconium dioxide support,
      a solvent,
   to obtain a reaction medium,
   heating of said reaction medium,
      to obtain ethylene glycol.

According to a particular embodiment, the invention relates to a method of preparation of ethylene glycol as defined above, wherein:
the hydrogenation step comprises
   bringing into contact:
      said oxalate compound,
      dihydrogen,
      said supported catalyst of formula CoCu/ZrO₂, comprising copper and cobalt on zirconium dioxide support,
      a solvent,
   to obtain a reaction medium put under a pressure,
   heating of said reaction medium
      to obtain ethylene glycol.

According to a particular embodiment, the invention relates to a method of preparation of ethylene glycol as defined above, wherein said oxalate is selected from dimethyloxale, diethyloxalate, dibenzyloxalate, diterbutyloxalate, diisopropyloxalate, diphenyloxalate.

According to a particular embodiment, the invention relates to a method of preparation of ethylene glycol as defined above, wherein said oxalate is selected from dimethyloxalate and diethyloxalate, preferably diethyloxalate.

Advantageously, the oxalates used can be obtained by carbonylation reaction of an alcohol in the presence of CO and oxygen.

Advantageously, carbon monoxide CO for the preparation of oxalate is produced by the electrolysis of carbon dioxide CO₂ into carbon monoxide CO.

Thus, the synthesis of ethylene glycol is advantageously carried out from the recovery of CO₂ and an alcohol.

Ethylene Glycol Preparation Method with Conditions on the Catalyst Used

According to a particular embodiment, the invention relates to a method of preparation of ethylene glycol as defined above, wherein said catalyst is according to the catalyst of the invention as defined above.

According to a particular embodiment, the invention relates to a method of preparation of ethylene glycol as defined above, wherein said catalyst is prepared by mixing the support in powder form with an aqueous solution of copper and cobalt salts, followed by calcination.

According to a particular embodiment, the invention relates to a method of preparation of ethylene glycol as defined above, wherein said catalyst is prepared by mixing the support in powder form with an aqueous solution of copper and cobalt salts, without the addition of additives or surfactants, followed by calcination.

Advantageously, the catalyst used in the method as defined above is prepared according to a preparation method comprising the following steps:
a step A of impregnating a cobalt salt and a copper salt, dissolved in an aqueous solution, in a volume of water from 5 to 10 mL, on a zirconium dioxide support in powder form, with a ratio of solution mass to support mass from 0.6 to 1.0;
to obtain the CoCu/ZrO₂ catalyst in the form of a homogeneous mixture, a step B of drying said homogeneous mixture, to obtain the CoCu/ZrO₂ catalyst in the form of a dry homogeneous mixture,
an activation step C, comprising calcination of said dry homogeneous mixture to obtain said catalyst.

Advantageously, in step A of impregnation, said aqueous solution containing the cobalt and copper salts is devoid of additives and surfactants. In other words, said aqueous impregnation solution consists of a demineralized solution of water in which the cobalt and copper salts are dissolved.

Advantageously, cobalt salt is cobalt nitrate and copper salt is copper nitrate.

The method of preparation of ethylene glycol as defined above according to the invention is therefore implemented with a catalyst prepared with an aqueous solution, advantageously free of additives and surfactants, which makes it more industrializable and at lower cost.

According to a particular embodiment, the invention relates to a method of preparation of ethylene glycol as defined above, wherein said catalyst has a surface area, analysed by BET, from 1 to 250 m²/g.

According to a particular embodiment, the invention relates to a method of preparation of ethylene glycol as defined above, wherein said catalyst has a surface area, analysed by BET, from 1 to 50 m²/g.

According to a particular embodiment, the invention relates to a method of preparation of ethylene glycol as defined above, wherein said catalyst has a surface area, analysed by BET, from 1 to 10 m²/g, preferably about 5 m²/g.

According to a particular embodiment, the invention relates to a method of preparation of ethylene glycol as defined above, wherein said catalyst comprises a crystalline phase, analysed by X-ray diffraction, crystallized in a monoclinic crystalline system.

According to a particular embodiment, the invention relates to a method of preparation of ethylene glycol as defined above, wherein said crystalline phase of the catalyst represents from 50 to 90% by total weight of the catalyst.

According to a particular embodiment, the invention relates to a method of preparation of ethylene glycol as defined above, wherein said crystalline phase is baddeleyite. According to a particular embodiment, the invention relates to a method of preparation of ethylene glycol as defined above, wherein the crystallite size of said crystalline phase is from 15 to 40 nm.

According to a particular embodiment, the invention relates to a method of preparation of ethylene glycol as defined above, wherein said zirconium dioxide support of said CoCu/ZrO₂ catalyst comprises baddeleyite in an amount from 50 to 90% by total weight of the catalyst.

The range from 50 to 90% includes the following ranges: from 50 to 55%, from 55 to 60%, from 60 to 65%, from 65 to 70%, from 70 to 75%, from 75 to 80%, from 80 to 85%, from 85 to 90%.

According to a particular embodiment, the invention relates to a method of preparation of ethylene glycol as defined above, wherein said catalyst comprises a total content of the bimetallic elements copper and cobalt from 0.5 to 25% by total weight of the catalyst. According to a particular embodiment, the invention relates to a method of preparation of ethylene glycol as defined above, wherein said catalyst comprises a total content of the bimetallic elements copper and cobalt from 5 to 20%.

According to a particular embodiment, the invention relates to a method of preparation of ethylene glycol as defined above, wherein the total content of the element copper is from 1 to 25% by total weight of the catalyst, preferably from 1 to 10% by total weight of the catalyst. According to a particular embodiment, the invention relates to a method of preparation of ethylene glycol as defined above, wherein the total content of the element cobalt is from 1 to 25% by total weight of the catalyst, preferably from 1 to 10% by total weight of the catalyst. In a particular embodiment, the invention relates to a method of preparation of ethylene glycol as defined above, wherein the mass quantity of cobalt is greater than that of copper.

According to a particular embodiment, the invention relates to a method of preparation of ethylene glycol as defined above, wherein the ratio by weight between copper and cobalt varies from 1:1 to 1:10, in particular from 1:1 to 1:5.

According to a particular embodiment, the invention relates to a method of preparation of ethylene glycol as defined above, wherein the mass composition of cobalt and copper of the catalyst is selected from Co(10%)Cu(5%) and Co(15%)Cu(5%).

According to a particular embodiment, the invention relates to a method of preparation of ethylene glycol as defined above, wherein the mass quantity of copper is greater than that of cobalt.

According to a particular embodiment, the invention relates to a method of preparation of ethylene glycol as defined above, wherein said catalyst has a ratio by weight between copper and cobalt varying from 1:1 to 1:10.

According to a particular embodiment, the invention relates to a method of preparation of ethylene glycol as defined above, wherein said catalyst has a ratio by weight between copper and cobalt varying from 2:10 to 5:10

According to a particular embodiment, the invention relates to a method of preparation of ethylene glycol as defined above, wherein the mass composition of cobalt and copper of the catalyst is selected from:
Co(2%)Cu(10%),
Co(5%)Cu(10%),
Co(5%)Cu(5%),
and Co(10%)Cu(10%),
   preferably Co(2%)Cu(10%) and Co(5%)Cu(10%),
   more preferentially Co(5%)Cu(10%).

According to a particular embodiment, the invention relates to a method of preparation of ethylene glycol as defined above, wherein said catalyst comprises a total content of the bimetallic elements copper and cobalt from 0.5 to 25% by total weight of the catalyst and in which the ratio by weight between cobalt to copper varies from 1:1 to 1:5, in particular, the mass composition of cobalt and copper of the catalyst is selected from:
Co(2%)Cu(10%),
Co(5%)Cu(10%),
Co(5%)Cu(5%),
and Co(10%)Cu(10%),
   preferably Co(2%)Cu(10%) and Co(5%)Cu(10%),
   more preferentially Co(5%)Cu(10%).

According to a particular embodiment, the invention relates to a method of preparation of ethylene glycol as defined above, wherein the mass composition of cobalt and copper of the catalyst is selected from Co(2%)Cu(10%) or Co(5%)Cu(10%), more preferentially Co(5%)Cu(10%), wherein the mass quantity of copper is greater than the mass content of cobalt.

According to a particular embodiment, the invention relates to a method of preparation of ethylene glycol as defined above, wherein the mass composition of cobalt and copper of the catalyst is Co(5%)Cu(10%) and the specific surface area is from 1 to 10 m²/g.

According to a particular embodiment, the invention relates to a method of preparation of ethylene glycol as defined above, wherein the mass composition of cobalt and copper of the catalyst varies from Co(2%)Cu(10%) to Co(10%)Cu(10%).

According to a particular embodiment, the invention relates to a method of preparation of ethylene glycol as defined above, wherein in said catalyst:
the total content of the element copper is from 5 to 10% by total weight of the catalyst, in particular 10% by total weight of the catalyst,
and the total content of the element cobalt is from 2 to 10% by total weight of the catalyst, in particular 5% by total weight of the catalyst.

According to a particular embodiment, the invention relates to a method of preparation of ethylene glycol as defined above, wherein the mass composition of cobalt and copper of the catalyst varies from Co(2%)Cu(10%) to Co(5%)Cu(10%).

According to a particular embodiment, the invention relates to a method of preparation of ethylene glycol as defined above, wherein in said catalyst:
the total content of the element copper is 10% by total weight of the catalyst,
and the total content of the element cobalt is from 2 to 5% by total weight of the catalyst, in particular 5% by total weight of the catalyst.

According to a particular embodiment, the invention relates to a method of preparation of ethylene glycol as defined above, wherein in said catalyst:
the total content of the element cobalt is from 2 to 5% by total weight of the catalyst, in particular 5% by total weight of the catalyst,
and the specific surface area of the catalyst is from 1 to 50 m²/g, preferably from 1 to 10 m²/g, analysed by BET.

According to a particular embodiment, the invention relates to a method of preparation of ethylene glycol as defined above, wherein in said catalyst:
the total content of the element cobalt is from 2 to 5% by total weight of the catalyst, in particular 5% by total weight of the catalyst,
the specific surface area of the catalyst is from 1 to 50 m²/g, preferably from 1 to 10 m²/g, analysed by BET,
and said catalyst comprises a crystalline phase, analysed by X-ray diffraction, crystallized in a monoclinic crystalline system, in particular baddeleyite, the crystallite size of which is preferably from 15 to 100 nm, in particular from 15 to 50 nm, in particular comprising Hafnium, Rhenium and Silicon impurities.

According to a particular embodiment, the invention relates to a method of preparation of ethylene glycol as defined above, wherein said catalyst possesses a molar amount of more than 50% of the element copper at oxidation degree (II) and/or of the element cobalt at oxidation degree (II).

According to a particular embodiment, the invention relates to a method of preparation of ethylene glycol as defined above, wherein said catalyst has a molar amount of more than 50% of the element copper at oxidation degree (II) and more than 50% of the element cobalt at oxidation degree (II).

According to a particular embodiment, the invention relates to a method of preparation of ethylene glycol as defined above, wherein said catalyst has a molar amount of more than 70% of the element copper at oxidation degree (II) and more than 65% of the element cobalt at oxidation degree (II).

According to a particular embodiment, the invention relates to a method of preparation of ethylene glycol as defined above, wherein said catalyst has a molar amount of more than 50% of the element copper at oxidation degree (II) in the form of CuO and more than 50% of the element cobalt at oxidation degree (II) in the form of CO₂O₃ or Co(OH)₂.

According to a particular embodiment, the invention relates to a method of preparation of ethylene glycol as defined above, wherein said catalyst has a molar amount of more than 70% of the element copper at oxidation degree (II) in the form of CuO and a molar amount of more than 65% of the element cobalt at oxidation degree (II) in the form of Co₂O₃ or Co(OH)₂. According to a particular embodiment, the invention relates to a method of preparation of ethylene glycol as defined above, wherein said catalyst has:
a molar amount from 10 to 30% of the element copper at oxidation degree (I) in the form of Cu₂O.

According to a particular embodiment, the invention relates to a method of preparation of ethylene glycol as defined above, wherein said catalyst has:
a molar amount from 10 to 30% of the element copper in the metallic state at oxidation degree (0) or in oxidation degree (I) in the form of Cu₂O
and a molar amount from 10 to 35% of the element cobalt in the metallic state at oxidation degree (0).

According to a particular embodiment, the invention relates to a method of preparation of ethylene glycol as defined above, wherein said catalyst has:
a molar amount greater than 70% of the element copper at oxidation degree (II) in the form of CuO,
a molar amount from 10 to 30% of the element copper at oxidation degree (I) in the form of Cu₂O
and a molar amount greater than 65% of the element cobalt at oxidation degree (II) in the form of Co₂O₃ or Co(OH)₂.

According to a particular embodiment, the invention relates to a method of preparation of ethylene glycol as defined above, wherein said catalyst has:
a molar amount greater than 70% of the element copper at oxidation degree (II) in the form of CuO,
a molar amount of 10 to 30% of the element copper in the metallic state at oxidation degree (0) or in oxidation degree (1) in the form of Cu₂O
a molar amount of 10 to 35% of the element cobalt in the metallic state at oxidation degree (0),
and a molar amount greater than 65% of the element cobalt at oxidation degree (II) in the form of Co₂O₃ or Co(OH)₂.

According to a particular embodiment, the invention relates to a method of preparation of ethylene glycol as defined above, wherein said catalyst has:
a molar amount of more than 50% of the element copper at oxidation degree (II) and of more than 50% of the element cobalt at oxidation degree (II),
a specific surface area from 1 to 50 m²/g
a crystalline phase, analysed by X-ray diffraction, crystallized in a monoclinic crystal system. According to a particular embodiment, the invention relates to a method of preparation of ethylene glycol as defined above, wherein said catalyst has:
   a molar amount greater than 70% of the element copper at oxidation degree (II) in the form of CuO,
   a molar amount from 10 to 30% of the element copper in the metallic state at oxidation degree (0) or in oxidation degree (I) in the form of Cu₂O
   a molar amount greater than 65% of the element cobalt at oxidation degree (II) in the form of Co₂O₃ or Co(OH)₂
   a specific surface area from 1 to 50 m²/g, in particular from 1 to 10 m²/g analysed by BET,
   and a crystalline phase, analysed by X-ray diffraction, crystallized in a monoclinic crystal system, notably baddeleyite, in particular comprising Hafnium, Rhenium and Silicon impurities.

According to a particular embodiment, the invention relates to a method of preparation of ethylene glycol as defined above, wherein said catalyst has:
a molar amount greater than 70% of the element copper at oxidation degree (II) in the form of CuO,
a molar amount from 10 to 30% of the element copper in the metallic state at oxidation degree (0) or in oxidation degree (I) in the form of Cu₂₀
a molar amount from 10 to 35% of the element cobalt in the metallic state at oxidation degree (0),
a molar amount greater than 65% of the element cobalt at oxidation degree (II) in the form of Co₂O₃ or Co(OH)₂.
a specific surface area from 1 to 50 m²/g, in particular from 1 to 10 m²/g analysed by BET,
and a crystalline phase, analysed by X-ray diffraction, crystallized in a monoclinic crystal system, notably baddeleyite, in particular comprising Hafnium, Rhenium and Silicon impurities.

According to a particular embodiment, the invention relates to a method of preparation of ethylene glycol as defined above, wherein said catalyst is in the form of micrometric particles from 1 to 500 μm.

### Ethylene Glycol Preparation Method Conditions

According to a particular embodiment, the invention relates to a method of preparation of ethylene glycol as defined above, wherein said catalyst is used in a proportion from 0.1 to 10 mmol of copper, in particular in a proportion of 4 mmol of Cu.

The range from 0.1 to 10 mmol includes the following ranges: 0.1 to 0.5 mmol, 0.5 to 1 mmol, 1 to 2 mmol, 2 to 3 mmol, 3 to 4 mmol.

According to a particular embodiment, the invention relates to a method of preparation of ethylene glycol as defined above, wherein the oxalate compound is used in a proportion from 2 to 400 molar equivalents, in particular in a proportion of 5 equivalents, relative to the metal Cu.

The range from 2 to 400 molar equivalents (eq.) includes the following ranges: 2 to 3 eq., 3 to 4 eq., 4 to 5 eq., 5 to 10 eq., 10 to 50 eq., 50 to 100 eq., 100 to 200 eq., 200 to 300 eq., 300 to 400 eq.

According to a particular embodiment, the invention relates to a method of preparation of ethylene glycol as defined above, wherein hydrogen is used under a pressure from 2 to 10 MPa, in particular 5 MPa.

The expression MPa corresponds to 10⁶ Pascal and is equivalent to 10 bars.

The expression “from 2.0 to 10.0 MPa” corresponds to the ranges: from 2.0 to 2.5 MPa; 2.5 to 3.0 MPa; 3.0 to 3.5 MPa; 3.5 to 4.0 MPa; 4.0 to 4.5 MPa; 4.5 to 5.0 MPa; 5.0 to 5.5 MPa; 5.5 to 6.0 MPa; 6.0 to 6.5 MPa; 6.5 to 7.0 MPa; 7.0 to 7.5 MPa; 7.5 to 8.0 MPa; 8.0 to 8.5 MPa; 8.5 to 9.0 MPa; 9.0 to 9.5 MPa; 9.5 to 10 MPa.

According to a particular embodiment, the invention relates to a method of preparation of ethylene glycol as defined above, wherein the solvent is selected from ethanol, methanol, isopropanol, tetrahydrofuran (THF), 2-methyltetrahydrofuran, toluene and dioxane, in particular ethanol.

According to a particular embodiment, the invention relates to a method of preparation of ethylene glycol as defined above, wherein the solvent is ethanol and the oxalate is diethyloxalate.

According to a particular embodiment, the invention relates to a method of preparation of ethylene glycol as defined above, wherein the reaction medium is placed under a pressure from 2 to 10 MPa, in particular 5 MPa.

According to a particular embodiment, the invention relates to a method of preparation of ethylene glycol as defined above, wherein the reaction medium is heated to a temperature from 100 to 250° C., in particular from 200 to 220° C.

The range from 100 to 250° C. includes the following ranges: from 100 to 150° C., 150 to 200° C. and 200 to 250° C.

According to a particular embodiment, the invention relates to a method of preparation of ethylene glycol as defined above, wherein the reaction medium is heated for 5 to 24 hours, in particular from 8 to 16 hours.

### Selectivity and Efficiency.

According to a particular embodiment, the invention relates to a method of preparation of ethylene glycol as defined above, wherein the hydrogenation reaction of said oxalate compound by hydrogen to obtain ethylene glycol is selective, with a selectivity of more than 70%, in particular more than 80%, preferably more than 90%, more preferably greater than or equal to 95%.

According to a particular embodiment, the invention relates to a method of preparation of ethylene glycol as defined above, wherein the hydrogenation reaction of said oxalate compound by hydrogen to obtain ethylene glycol has a yield greater than 50%, in particular greater than 70%, preferably greater than 80%.

According to a particular embodiment, the invention relates to a method of preparation of ethylene glycol as defined above, wherein the catalyst is recovered at the end of a hydrogenation reaction step and is reused as a catalyst in another catalysed reaction step.

According to a particular embodiment, the invention relates to a method of preparation of ethylene glycol as defined above, wherein the catalyst is recovered at the end of a reaction step of hydrogenation and is reused as a catalyst in another subsequent reaction step of hydrogenation.

Advantageously, the catalyst recovered at the end of a hydrogenation reaction step can be used for several successive cycles of hydrogenation reaction steps.

According to a particular embodiment, the invention relates to a method of preparation of ethylene glycol as defined above,
wherein the hydrogen is used under a pressure from 2 to 10 MPa, in particular 6 MPa,
wherein the reaction medium is heated to a temperature from 100 to 250° C., in particular from 180 to 220° C., optionally wherein the reaction medium is heated for 5 to 24 hours, in particular for 8 or 16 hours,
wherein during said heating step of the reaction medium, said supported bimetallic catalyst is activated, said activated catalyst consisting of:
   from 80 to 100%, in particular from 95 to 100%, preferably 100%, of metallic copper atoms at oxidation degree zero Cu(0) and
   from 80 to 100% of cobalt atoms at an oxidation degree greater than zero.

Advantageously, the embodiments of the method according to the invention do not require prior activation of the catalyst by reduction of metal species, the catalyst being activated in-situ during the process.

The catalytic species of the oxalate hydrogenation reaction, i.e. metallic copper with oxidation degree (0), are formed in-situ. The second metallic element, cobalt, mainly in oxidized form on the surface of the support, plays a role as a promoter on copper for the catalysis of the oxalate hydrogenation reaction. Thus, the bimetallic catalyst based on copper and cobalt can be introduced directly into the oxalate hydrogenation method according to the invention and it does not need to be reduced to metal form beforehand to initiate the hydrogenation reaction, unlike many bimetallic catalysts of the prior art which are inactive in the absence of a first step of reduction of the catalytic species.

### Flow Method Conditions

The method according to the invention may be implemented in a flow chemistry apparatus, e.g. in commercial reactors such as “H-Cube Pro®” or “Phoenix®” of ThalesNano INC. (7 Zahony Street, Graphisoft Park, Building D, H-1031 Budapest, Hungary) or such as the “E-Series” or “R-Series flow chemistry systems” reactors of Vapourtec Ltd (Unit 21/Park Farm Business Centre/Fornham Pk, Bury Saint Edmunds IP28 6TS, UK).

Advantageously, the continuous flow method is carried out at a temperature from 100° C. to 250° C.

Advantageously, the continuous flow method is carried out at a pressure from 0.1 MPa to 10 MPa, preferably 5 MPa.

According to a particular embodiment, the continuous flow method is carried out in a reactor in which gases represent from 10 to 90% of the reactor volume.

The expression “10 to 90%” corresponds to the following ranges: from 10 to 20%; from 20 to 30%; from 30 to 40%; from 40 to 50%; from 50 to 60%; from 60 to 70%; from 70 to 80%; from 80 to 90%.

According to a particular embodiment, the continuous flow method is carried out by means allowing a contact time between the reagents from 1 second to 2 hours, in particular from 1 second to 2 minutes.

The expression “from 1 second to 2 hours” corresponds to the scales: from 1 to 15 seconds; from 15 to 30 seconds; from 30 seconds to 1 minute; from 1 to 2 minutes; from 2 to 15 minutes; from 15 to 30 minutes; from 30 minutes to 1 hour; from 1 to 2 hours.

According to a particular embodiment, the reaction step of hydrogenation of the method is carried out in a continuous flow and includes means for introducing into the reactor the hydrogen flow in contact with the substrate (oxalate).

According to a particular embodiment, the invention relates to a method of preparation of ethylene glycol as defined above, wherein oxalate circulates under flow and/or dihydrogen circulates under flow, through or in contact with said catalyst.

### Catalyst Preparation Method

A fourth object of the invention relates to a method of preparation of a CoCu/ZrO₂ catalyst according to the catalyst of the invention as defined above, comprising:
a step A of impregnating a cobalt salt and a copper salt, dissolved in an aqueous solution, free of additives and surfactants, in a volume of water from 5 to 10 mL, on a zirconium dioxide support in powder form, with a ratio of solution mass to support mass from 0.6 to 1.0;
to obtain the CoCu/ZrO₂ catalyst in the form of a homogeneous mixture, said support having
   a surface area from 1 to 250 m²/g, notably from 1 to 75 m²/g, in particular from 1 to 50 m²/g, preferably from 1 to 5 m²/g
   and comprising a crystalline phase, analysed by X-ray diffraction, crystallized in a monoclinic crystalline system,
a step B of drying said homogeneous material, at a temperature from 60 to 100° C., for a period from 10 to 24 hours, to obtain the CoCu/ZrO₂ catalyst in the form of a dry homogeneous mixture,
an activation step C, comprising calcination under air of said dry homogeneous mixture at a temperature from 200 to 1000° C., for a period from 1 to 15 hours, to obtain said catalyst.

According to a particular embodiment, the invention relates to a method as defined above for the preparation of a catalyst according to the invention, wherein in step A, said cobalt salt is cobalt nitrate and/or said copper salt is copper nitrate.

According to a particular embodiment, the invention relates to a method as defined above for the preparation of a catalyst according to the invention, wherein in step A, said support is used in an amount from 1 to 50 grams.

According to a particular embodiment, the invention relates to a method as defined above for the preparation of a catalyst according to the invention, wherein in step A, said support used has a surface area, analysed by BET, from 1 to 75 m²/g.

According to a particular embodiment, the invention relates to a method as defined above for the preparation of a catalyst according to the invention, wherein in step A, said support used has a surface area, analysed by BET, from 1 to 50 m²/g.

According to a particular embodiment, the invention relates to a method as defined above for the preparation of a catalyst according to the invention, wherein in step A, said catalyst support used has a surface area, analysed by BET, from 1 to 10 m²/g, preferably from 5 to 7 m²/g.

According to a particular embodiment, the invention relates to a method as defined above for the preparation of a catalyst according to the invention, wherein in step A, said support used has a crystalline phase representing from 50 to 90% by total weight of the catalyst.

According to a particular embodiment, the invention relates to a method as defined above for preparing a catalyst according to the invention, wherein in step A, said support used comprises baddeleyite.

According to a particular embodiment, the invention relates to a method as defined above for the preparation of a catalyst according to the invention, wherein in step A, said support used comprises baddeleyite whose crystallite size is from 15 to 100 nm, preferably from 15 to 50 nm, and optionally comprising Hafnium (Hf), Rhenium (Re) and silicon (Si) impurities.

Advantageously, the percentage mass ratio between Hafnium atoms and zirconium atoms (Hf/Zr) is less than 5% in said support used.

Advantageously, the percentage mass ratio between Rhenium atoms and zirconium atoms (Re/Zr) is less than 5% in said support used.

Advantageously the percentage mass ratio between silicon atoms and zirconium atoms (Si/Zr) is less than 2% in said support used.

Advantageously in said ZrO₂ support:
the percentage mass ratio between Hafnium atoms and zirconium atoms (Hf/Zr) is less than 5%,
the percentage mass ratio between Rhenium atoms and zirconium atoms (Re/Zr) is less than 5%,
the percentage mass ratio between silicon atoms and zirconium atoms (Si/Zr) is less than 2%.

According to a particular embodiment, the invention relates to a method as defined above for the preparation of a catalyst according to the invention, wherein in step A, the total mass content of the bimetallic elements copper and cobalt is from 0.5 to 25% by total weight of the catalyst.

According to a particular embodiment, the invention relates to a method as defined above for the preparation of a catalyst according to the invention, wherein in step A, the total mass content of the bimetallic elements copper and cobalt is from 5 to 20% by total weight of the catalyst.

According to a particular embodiment, the invention relates to a method as defined above for the preparation of a catalyst according to the invention, wherein in step A, the total mass content of the element copper is from 1 to 25% by total weight of the catalyst, preferably from 1 to 10% by total weight of the catalyst.

According to a particular embodiment, the invention relates to a method as defined above for preparing a catalyst according to the invention, wherein in step A, the total mass content of the element cobalt is from 1 to 25% by total weight of the catalyst, preferably from 1 to 10% by total weight of the catalyst.

According to a particular embodiment, the invention relates to a method as defined above, wherein the mass quantity of cobalt is greater than that of copper.

According to a particular embodiment, the invention relates to a method as defined above, wherein in step A, the ratio by weight between copper and cobalt varies from 1:1 to 1:10, in particular from 1:1 to 1:5.

According to a particular embodiment, the invention relates to a method as defined above, wherein in step A, the mass composition cobalt and copper of the catalyst is selected from Co(10%)Cu(5%) and Co(15%)Cu(5%).

According to a particular embodiment, the invention relates to a method as defined above, wherein the mass quantity of copper is greater than that of cobalt.

According to a particular embodiment, the invention relates to a method as defined above for preparing a catalyst according to the invention, wherein in step A, the ratio by weight between cobalt and copper varies from 1:1 to 1:10.

According to a particular embodiment, the invention relates to a method as defined above for preparing a catalyst according to the invention, wherein in step A, the weight ratio between cobalt and copper varies from 2:10 to 5:10.

According to a particular embodiment, the invention relates to a method as defined above for preparing a catalyst according to the invention, wherein in step A, the support is in the form of micrometric particles from 1 to 500 μm.

According to a particular embodiment, the invention relates to a method as defined above for the preparation of a catalyst according to the invention, wherein in step B, said homogeneous mixture is dried at 80° C. for 16 hours.

According to a particular embodiment, the invention relates to a method as defined above for preparing a catalyst according to the invention, wherein in step C, said dry homogeneous mixture is calcined at 600° C. for 2 hours.

### Catalyst Prepared According to the Method of the Invention

A fifth object of the present invention relates to a supported bimetallic catalyst comprising copper and cobalt on a zirconium dioxide support, of the formula CoCu/ZrO₂, obtainable by the method of the preparation of a catalyst as defined above.

### Characteristics of the Catalyst in a Reducing Atmosphere

According to a particular embodiment, the invention relates to a supported bimetallic catalyst prepared according to the invention and as defined above,
wherein said catalyst analysed by temperature-programmed reduction (TPR) carried out in a dihydrogen reducing atmosphere over a temperature range from 30 to 900° C., is characterized by a reduction temperature of metallic copper of oxidation degree (0) in the range from 150 to 250° C., in particular from 180 to 220° C.

Advantageously, at a temperature higher than said reduction temperature of metallic copper, in particular by at least 10° C., copper atoms are from 80 to 100%, in particular from 95 to 100%, preferably 100%, in metallic form.

Advantageously, at a temperature higher than said reduction temperature of metallic copper, in particular by at least 10° C., cobalt atoms are at more than 80%, in particular from 80 to 100%, preferably 100% in oxidized form, i.e. at an oxidation degree greater than zero.

Advantageously, at a temperature higher than said reduction temperature of metallic copper, in particular by at least 10° C., cobalt atoms are from 0 to 20%, in particular from 0 to 10%, preferably less than 5%, in metallic form at oxidation degree zero.

According to a particular embodiment, the invention relates to a supported bimetallic catalyst as defined above, wherein said catalyst analysed by temperature-programmed reduction (TPR) carried out in a dihydrogen reducing atmosphere over a temperature range from 30 to 900° C., is characterized by a reduction temperature of metallic copper of oxidation degree (0) in the range from 150 to 250° C.,
in particular at a temperature greater than said reduction temperature of metallic copper by at least 10° C. analysed by TPR,
copper atoms are from 80 to 100%, preferably 100% in metallic form,
cobalt atoms are from 80 to 100%, preferably 100% in oxidized form.

According to a particular embodiment, the invention relates to a supported bimetallic catalyst as defined above, wherein said catalyst analysed by temperature-programmed reduction (TPR) carried out in a reducing atmosphere under a flux of 5 vol. % H₂ in Argon, in particular at 30 mL/min, over a temperature range from 30 to 900° C., in particular with a heating rate of 5° C./min, is characterized by a reduction temperature of metallic copper of oxidation degree (0) in the range from 150 to 250° C.

Advantageously, the catalyst is pre-treated in an inert atmosphere before the said analysis, in particular at 200° C. under Helium.

According to a particular embodiment, the invention relates to a supported bimetallic catalyst as defined above, wherein said catalyst under a reducing atmosphere of hydrogen at a pressure from 2 to 10 MPa and at a temperature of 150 to 250° C., in particular from 180 to 220° C., comprises:
from 80 to 100%, in particular from 95 to 100%, preferably 100%, of metallic copper atoms Cu(0) and
from 80 to 100% of cobalt atoms at an oxidation degree greater than zero.

Advantageously copper atoms are 95%, preferably 100% in metallic form.

Advantageously cobalt atoms are from 80 to 100%, in particular from 95 to 100%, preferably 100%, in oxidized form at an oxidation degree greater than zero.

Advantageously cobalt atoms are from 0 to 20%, in particular from 0 to 15%, preferably less than 20%, in metallic form.

### Catalyst in Activated Form

Another object of the present invention relates to an activated supported bimetallic catalyst, comprising copper and cobalt on the surface, on a zirconium dioxide support, of the formula CuCo/ZrO₂, wherein
copper atoms are from 80 to 100%, in particular from 95 to 100%, preferably 100%, at oxidation degree zero Cu(0) and
cobalt atoms are from 80 to 100%, in particular from 95 to 100%, preferably 100%, at an oxidation degree greater than zero.

The term “activated catalyst” means the catalyst in its activated form, i.e. its form during the chemical reaction catalysed by this catalyst, namely a form that includes the catalytic species at the origin of the catalysis.

### Use of the Catalyst for the Hydrogenolysis of Ethylene Glycol or to Depolymerize Biomass

Another object of the present invention relates to the use of a CoCu/ZrO₂ catalyst according to the invention as defined above, for the hydrogenolysis of ethylene glycol.

The inventors surprisingly observed that after the hydrogenation reaction of oxalates into ethylene glycol, when oxalate is no longer present as a substrate in the medium, prolonged contact of ethylene glycol with the CoCu/ZrO₂ catalyst according to the invention, leads to hydrogenolysis of ethylene glycol. Advantageously, the hydrogenation reaction of oxalate to ethylene glycol and the hydrogenolysis reaction of ethylene glycol are subsequent and can be separated.

For the purposes of the present invention, “hydrogenolysis” means the cleavage reaction of at least one carbon-carbon C—C or carbon-heteroatom covalent bond such as the carbon-oxygen bond, by the action of hydrogen.

Another object of the present invention relates to a method for hydrogenolysis of ethylene glycol comprising:
a step of bringing into contact ethylene glycol in the presence of hydrogen and a heterogeneous supported bimetallic catalyst comprising copper and cobalt on a zirconium dioxide support, of formula CoCu/ZrO₂ as defined above.

According to a particular embodiment, the invention relates to a method for hydrogenolysis of ethylene glycol as defined above, comprising:
bringing into contact:
   ethylene glycol,
   dihydrogen, in particular from 20 to 100 bars, preferably at 60 bars
   said supported catalyst of the formula CoCu/ZrO₂, comprising cobalt and copper on a zirconium dioxide support,
   optionally a solvent, in particular tetrahydrofuran (THF),
   optionally a base, in particular potassium ter-butylate (KOtBu), to obtain a reaction medium, optionally pressurized,
optionally heating said reaction medium,
   to obtain degradation of ethylene glycol by cleavage of at least one carbon-carbon or carbon-oxygen covalent bond.

Another object of the present invention relates to the use of a CoCu/ZrO₂ catalyst according to the invention as defined above to depolymerize biomass.

According to a particular embodiment, the invention relates to the use of a CoCu/ZrO₂ catalyst as defined above to depolymerize biomass by hydrogenolysis of the ethylene glycol groups present in the compounds constituting said biomass.

The inventors surprisingly observed that the CoCu/ZrO₂ catalyst according to the invention could catalyse the hydrogenolysis reaction of the diol groups present in the carbohydrate molecules contained in the biomass by cleaving the C—C or C—O carbon-carbon bonds.

In the present invention, “biomass” means organic matter of plant origin (including microalgae), animal, bacterial or fungal (fungi), usable as a source of energy (bioenergy). Biomass includes carbohydrate molecules or polyols, in which diol groups —CHOH—CHOH—, containing C—C and C—O covalent bonds, are present.

For the purposes of the present invention, “depolymerize biomass” means the action of cleaving carbon-carbon C—C or carbon-heteroatom C—X covalent bonds such as the carbon-oxygen C—O bond, of carbohydrate molecules contained in biomass.

Another object of the invention relates to a method of degradation of biomass comprising:
a hydrogenolysis step of the C—C and C—O bonds in the diol groups contained in the carbohydrate molecules contained in the biomass, in the presence of a supported bimetallic catalyst comprising copper and cobalt on a zirconium dioxide support, of formula CoCu/ZrO₂ as defined above.

The following examples and figures illustrate the invention, without limiting its scope.

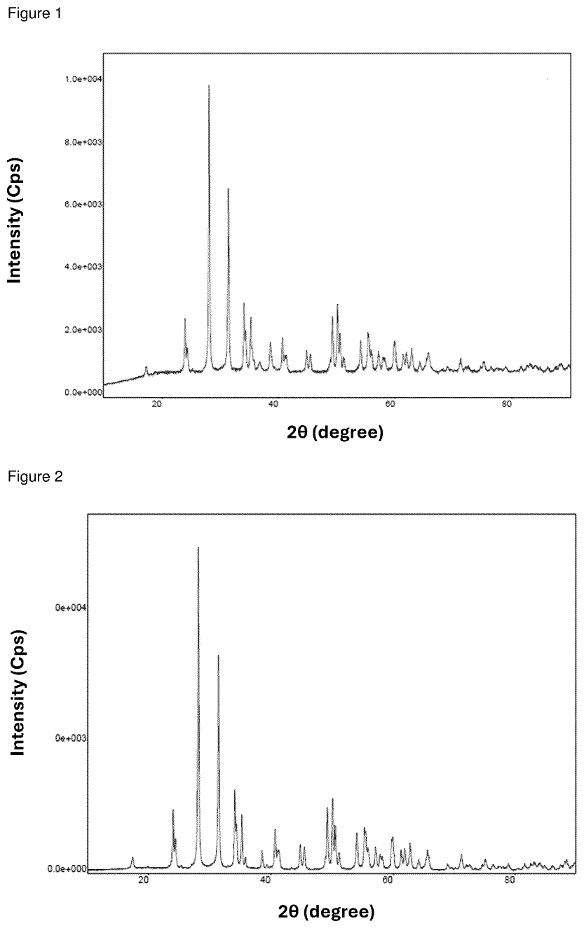

FIG. 1 shows the X-ray powder diffractogram of a Cu(10%) Co(5%)/ZrO₂(A) catalyst, with a specific surface area of about 5 m²/g prepared with a ZrO₂(A) support.

FIG. 2 shows the X-ray powder diffractogram of a ZrO₂(A) support which was calcined at 600° C. for 2 hours.

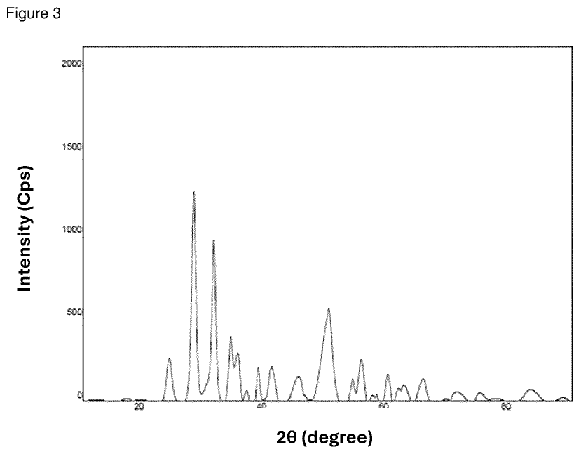

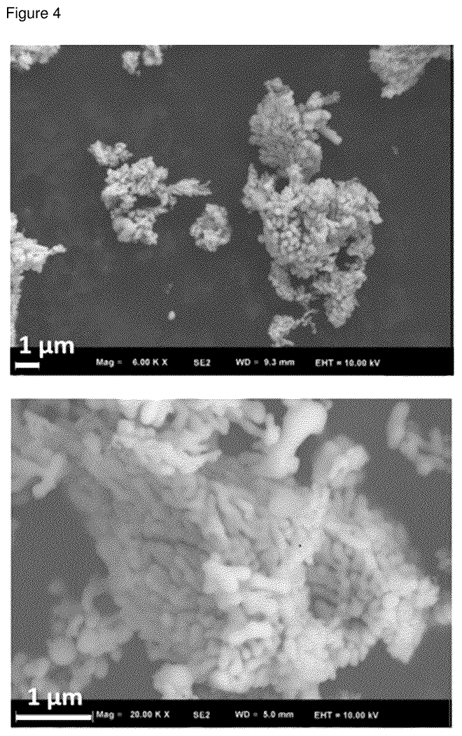

FIG. 3 shows the X-ray powder diffractogram of a Cu(10%) Co(5%)/ZrO₂ (B) catalyst, with a specific surface area of 59 m²/g.

FIG. 4 shows scanning electron microscopy images of a Cu(10%) Co(5%)/ZrO₂(A) catalyst, with a specific area of about 5 m²/g, calcined at 600° C. for 2 hours

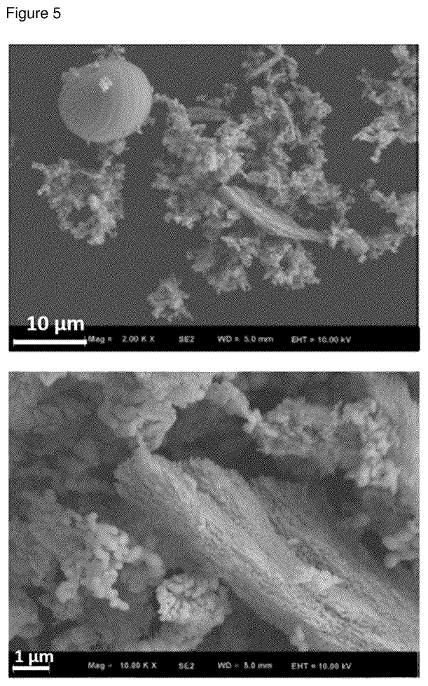

FIG. 5 shows scanning electron microscopy images of a ZrO₂(A) support calcined at 600° C. for 2 hours.

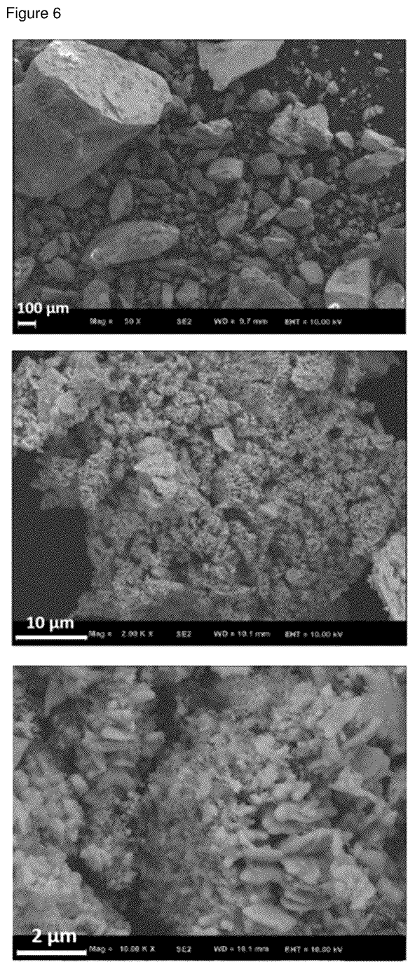

FIG. 6 shows scanning electron microscopy images of a Cu(10%) Co(5%)/ZrO₂ (B) catalyst, with a specific surface area of 59 m²/g, calcined at 600° C. for 2 hours,

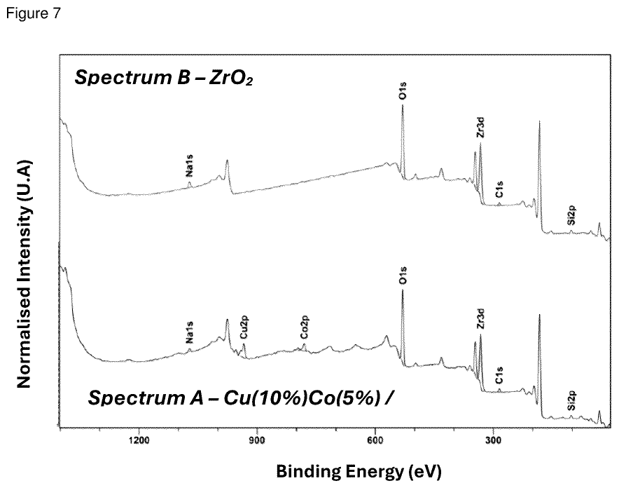

FIG. 7 shows the X-ray photoelectron spectrometry spectrum of a Cu—Co/ZrO₂(A) catalyst, calcined at 600° C. for 2 hours, with a composition of Cu at 10% and Co at 5% by weight relative to the total weight of the catalyst (spectrum A) and the X-ray photoelectron spectrometry spectrum of the support used ZrO₂(A) for the preparation of the said catalyst calcined at 600° C. for 2 hours (spectrum B).

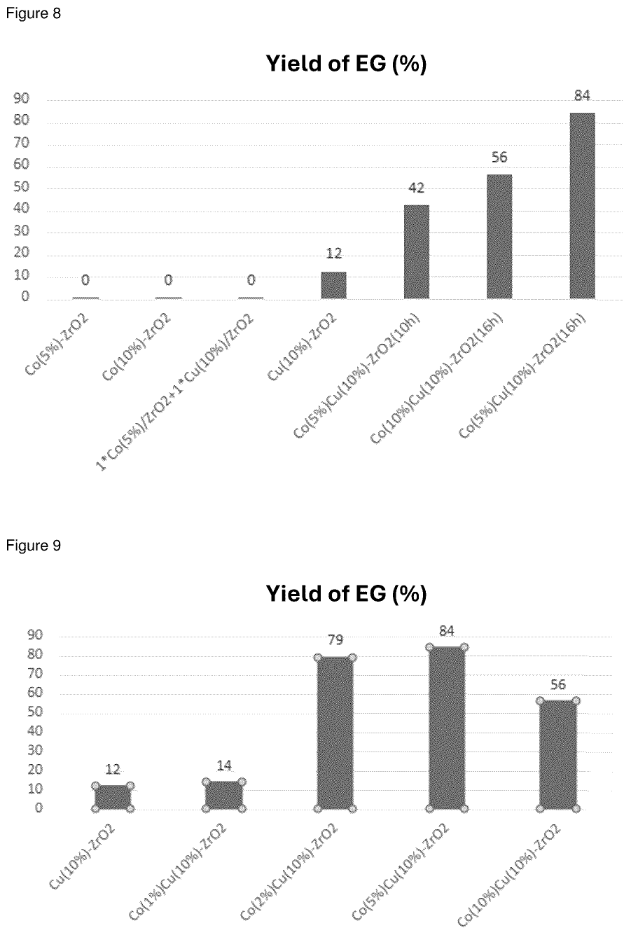

FIG. 8 is a histogram of ethylene glycol (EG) yields from tests carried out with monometallic copper or cobalt catalysts, bimetallic copper-cobalt catalysts, and catalyst-support mixtures.

FIG. 9 is a histogram of the ethylene glycol (EG) yields from tests carried out with 10% copper monometallic catalysts, copper-cobalt bimetallic catalysts at a constant 10% copper content and a variable cobalt content from 1 to 10%.

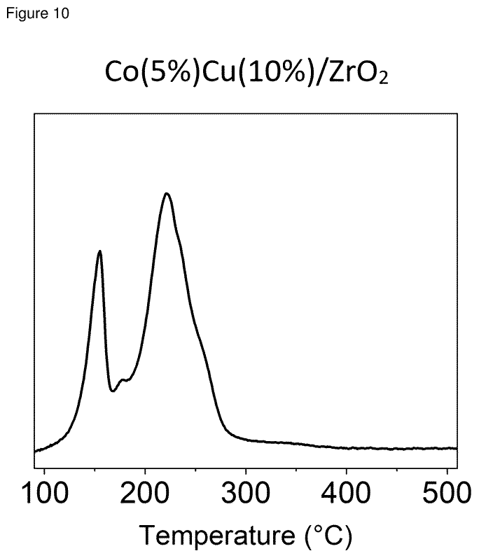

FIG. 10 represents the analysis curve of the temperature-programmed reduction under dihydrogen flux (H₂-TPR) of catalysts for the prepared Cu(10%) Co(5%)/ZrO₂ catalysts in the temperature range from 100 to 500° C.

### EXAMPLE 1—MATERIALS AND METHODS

Two zirconium dioxide supports ZrO₂(A) and ZrO₂ (B) of different specific surface area, respectively from 5 to 7 m²/g for ZrO₂(A) and of 85 m²/g for ZrO₂ (B), were used for the preparation of the catalysts.

The ZrO₂ zirconium dioxide support, called ZrO₂(A), with a specific surface area from 5 to 7 m²/g, is supplied by Sterm Chemicals (15 Rue de l'Atome, 67800 Bischheim) with the reference number 93-4013.

The ZrO₂(A) zirconium dioxide support supplied by Sterm Chemicals comprises approximately 97% zirconium dioxide, 1.86% Hafnium dioxide (HfO₂) by weight, and trace amounts of silica (SiO₂) and Yttrium oxide (Y₂O₃).

The ZrO₂ zirconium dioxide support, called ZrO₂ (B), with a specific surface area greater than 85 m²/g is supplied by Thermo Fisher Scientific (formerly Alfa Aesar) with the product number AA4381522.

The γ-Al₂O₃ supports are supplied by Strem Chemicals (15 Rue de l'Atome, 67800 Bischheim) with reference number 13-2525).

The SiO₂ support (40-63 mm) was supplied by VWR chemicals reference 154425P.

Cobalt nitrate (Co(NO₃)₂·3H₂O) and copper nitrate Cu(NO₃)₂·3H₂O were supplied by Fischer. The autoclave is supplied by Parr Instrument Company.

### EXAMPLE 2—GENERAL PROCEDURE FOR THE PREPARATION OF HETEROGENEOUS CO/ZRO₂ CATALYSTS

Cobalt nitrate Co(NO₃)₂·6H₂O was dissolved in a minimum volume of deionized water, from 5 to 10 mL, forming a solution. This solution containing the metal precursors was added to the appropriate amount of ZrO₂(A) zirconium dioxide support (with 5 to 7 m²/g of specific surface area measured by BET) and the resulting ZrO₂ paste was mixed at room temperature until a homogeneous material was obtained. The material was then dried at 80° C. for 16 hours, then calcined at 600° C. under air for 2 hours to obtain the catalyst.

### EXAMPLE 3—GENERAL PROCEDURE FOR THE PREPARATION OF HETEROGENEOUS CU/ZRO₂ CATALYSTS

Copper nitrate Cu(NO₃)₂·3H₂O was dissolved in a minimum volume of demineralized water, from 5 to 10 mL, forming a solution. This solution containing the metal precursors was added to the appropriate amount of ZrO₂(A) zirconium dioxide support (with 5 to 7 m²/g of specific surface area measured by BET) and the resulting ZrO₂ paste was mixed at room temperature until a homogeneous material was obtained. The material was then dried at 80° C. for 16 hours, then calcined at 600° C. under air for 2 hours to obtain the catalyst.

### EXAMPLE 4—GENERAL PROCEDURE FOR THE PREPARATION OF HETEROGENEOUS CU—CO/ZRO₂(A) CATALYSTS

Cobalt nitrate Co(NO₃)₂·6H₂O and copper nitrate Cu(NO₃)₂·3H₂O were dissolved in a minimum volume of demineralized water, from 5 to 10 mL forming a solution. This solution containing the metal precursors was added to the appropriate amount of ZrO₂(A) support with a specific surface area of 5 to 7 m²/g, and was mixed at room temperature until a homogeneous material was obtained. The material was then dried at 80° C. for 16 h and then calcined at 600° C. under air for 2 hours to obtain the catalyst.

### EXAMPLE 5—PREPARATION OF HETEROGENEOUS MONO- AND BIMETALLIC CATALYSTS CO/ZRO₂, CU/ZRO₂, CU—CO/ZRO₂ IN WHICH THE ZIRCONIUM DIOXIDE HAS A SPECIFIC SURFACE AREA OF 5 TO 7 M²/G

Table 1 below reports the preparation conditions for Co/ZrO₂, Cu/ZrO₂ and Cu—Co/ZrO₂ catalysts prepared according to examples 2, 3 and 4 with a support.

TABLE 1
Co/ZrO₂, Cu/ZrO₂ and Cu—Co/ZrO₂ catalysts prepared
with ZrO₂(A) with a specific surface area of 5 to 7 m²/g.

| | Support | | Co(NO₃)₂•6H₂O | Cu(NO₃)₂•3H₂O |
|---|---|---|---|---|
| Catalyst | (g) | | (mg) | (mg) |
| Cu(10%)/ZrO₂(A) | 10 | g | 0 | 3802 |
| Co(5%)/ZrO₂(A) | 5 | g | 1250 | 0 |
| Co(10%)/ZrO₂(A) | 5 | g | 2500 | 0 |
| Co(5%)—Cu(10%)/ | 40 | g | 10000 | 15200 |
| ZrO₂(A) | | | | |
| Co(1%)—Cu(10%)/ | 5 | g | 250 | 1900 |
| ZrO₂(A) | | | | |
| Co(2%)—Cu(10%)/ | 5 | g | 500 | 1900 |
| ZrO₂(A) | | | | |
| Co(5%)—Cu(10%)/ | 5 | g | 1250 | 1900 |
| ZrO₂(A) | | | | |
| Co(10%)—Cu(10%)/ | 5 | g | 2500 | 1900 |
| ZrO₂(A) | | | | |
| Co(5%)—Cu(5%)/ | 5 | g | 1250 | 950 |
| ZrO₂(A) | | | | |
| Co(5%)—Cu(2%)/ | 5 | g | 1250 | 380 |
| ZrO₂(A) | | | | |

### EXAMPLE 6—GENERAL PROCEDURE FOR THE PREPARATION OF CU—CO/SUPPORT HETEROGENEOUS CATALYSTS

Cobalt nitrate Co(NO₃)₂·6H₂O and copper nitrate Cu(NO₃)₂·3H₂O were dissolved in a minimum volume of demineralized water, from 5 to 10 mL forming a solution. This solution containing the metal precursors was added to the appropriate amount of support (ZrO₂ (B) with a specific surface area greater than 85 m²/g, SiO₂, CeO₂ or γ-Al₂O₃) and was mixed at room temperature until a homogeneous material was obtained. The material was then dried at 80° C. for 16 h and then calcined at 600° C. under air for 2 hours to obtain the catalyst.

Table 2 below reports the preparation conditions of a Cu—Co/Support catalyst prepared according to Example 6.

TABLE 2
Co—Cu/Support catalyst prepared with various supports.

| | Support | Co(NO₃)₂•6H₂O | Cu(NO₃)₂•3H₂O |
|---|---|---|---|
| Catalyst | (g) | (mg) | (mg) |
| Co(5%)—Cu(10%)/ | 5 g | 1250 | 1900 |
| ZrO₂(B) | | | |
| Co(5%)—Cu(10%)/SiO₂ | 5 g | 1250 | 1900 |
| Co(5%)—Cu(10%)/γ- | 5 g | 1250 | 1900 |
| Al₂O₃ | | | |

### EXAMPLE 7—GENERAL PROCEDURE FOR HETEROGENEOUS CATALYSIS OF THE HYDROGENATION OF DIALKYL OXALATE IN A 450 ML REACTOR

In a 450 mL Parr autoclave, equipped with a magnetic stirrer, a heterogeneous catalyst based on copper (4 mmol Cu) or based on copper (4 mmol Cu) and cobalt or based on cobalt (4 mmol Co), diethyl oxalate (20 mmol) and ethanol (50 mL) as a solvent were introduced. The reactor was sealed, and the reaction mixture was purged three times with nitrogen (5 bars), and twice with hydrogen (5 bars).

The autoclave was then pressurized with 50 bars of hydrogen. The reaction medium was then stirred at 200° C. for 16 hours or 220° C. for 8 hours.

Once the reaction was complete, the autoclave was brought back to room temperature before being depressurized and purged three times with nitrogen (5 bars).

The final mixture obtained was diluted in ethanol or methanol and then an internal standard (mesitylene) was added, to calculate the yield by using GC-MS.

### EXAMPLE 8—TESTS CARRIED OUT WITH CU/ZRO₂(A), CO/ZRO₂(A) AND COCU/ZRO₂(A) CATALYSTS

Table 3 reports the conditions of the hydrogenation tests with a Cu/ZrO₂(A), Co/ZrO₂(A) or CoCu/ZrO₂(A) catalyst prepared according to Example 5, as well as the yield (Yld) and selectivity (selec). Hydrogenation yield and selectivity are calculated by using GC-MS, mesitylene is used as the internal standard.

TABLE 3
Conditions of hydrogenation tests with a Cu/ZrO₂(A),
Co/ZrO₂(A) or CoCu/ZrO₂(A) catalyst and the obtained results.

| | | H₂ | T, | Time, | | V | Yield |
|---|---|---|---|---|---|---|---|
| Reaction | Cat | (bar) | (° C.) | (h) | n oxalate | solvent | (Selectivity) |
| A-1 | Cu(10%)—ZrO₂(A) | 50 | 200 | 16 | 20 mmol DEO | 50 mL | Yld = 12% |
| | (4 mmol) | | | | | éthanol | (selec: 13%) |
| | 20 mol % | | | | | | |
| A-2 | Co(5%)—ZrO₂(A) | 50 | 200 | 16 | 20 mmol DEO | 50 mL | Yld = 0% |
| | (4 mmol) | | | | | éthanol | |
| | 20 mol % | | | | | | |
| A-3 | Co(10%)—ZrO₂(A) | 50 | 200 | 16 | 20 mmol DEO | 50 mL | Yld = 0% |
| | (4 mmol) | | | | | éthanol | |
| | 20 mol % | | | | | | |
| A-4 | Co(1%)—Cu(10%)—ZrO₂(A) | 50 | 200 | 16 | 20 mmol DEO | 50 mL | Yld = 14% |
| | (4 mmol) | | | | | éthanol | (selec: 12%) |
| | 20 mol % | | | | | | |
| A-5 | Co(2%)—Cu(10%)—ZrO₂(A) | 50 | 200 | 16 | 20 mmol DEO | 50 mL | Yld = 79% |
| | (4 mmol) | | | | | éthanol | (selec: 95%) |
| | 20 mol % | | | | | | |
| A-6 | Co(10%)—Cu(10%)—ZrO₂(A) | 50 | 200 | 16 | 20 mmol DEO | 50 mL | Yld = 56% |
| | (4 mmol) | | | | | éthanol | (selec: 71%) |
| | 20 mol % | | | | | | |
| A-7 | Co(5%)—Cu(10%)—ZrO₂(A) | 50 | 200 | 16 | 20 mmol DEO | 50 mL | Yld = 84% |
| | (4 mmol) | | | | | éthanol | (selec: 95%) |
| | 20 mol % | | | | | | |
| A-8 | Co(5%)—Cu(10%)—ZrO₂(A) | 50 | 200 | 24 | 20 mmol DEO | 50 mL | Yld = 50% |
| | (4 mmol) | | | | | éthanol | |
| | 20 mol % | | | | | | |
| A-9 | Co(5%)—Cu(10%)—ZrO₂(A) | 50 | 220 | 8 | 20 mmol DEO | 50 mL | Yld = 74% |
| | (4 mmol) | | | | | éthanol | (selec: 85%) |
| | 20 mol % | | | | | | |
| A-10 | Co(5%)—Cu(5%)—ZrO₂(A) | 50 | 200 | 16 | 20 mmol DEO | 50 mL | Yld = 42% |
| | (4 mmol) | | | | | éthanol | (selec: 46%) |
| | 20 mol % | | | | | | |
| A-7/2 | Co(5%)—Cu(10%)—ZrO₂ | 50 | 200 | 16 | 20 mmol DEO | 50 mL | Yld = 70% |
| | (2 mmol) | | | | | éthanol | |
| | 10 mol % | | | | | | |

### EXAMPLE 9—TESTS CARRIED OUT WITH COCU CATALYSTS ON VARIOUS SUBSTRATES PREPARED ACCORDING TO EXAMPLE 6

Table 4 reports the conditions of the hydrogenation tests respectively with a CoCu catalyst on various substrates, prepared according to Example 6, as well as the yield (Yld) and selectivity (selec). Hydrogenation efficiency and selectivity are calculated by using GC-MS, mesitylene is used as the internal standard.

TABLE 4
Conditions of hydrogenation tests with a CoCu catalyst
on various supports and the obtained results.

| | | H₂ | T, | Time, | | | Yield |
|---|---|---|---|---|---|---|---|
| Reaction | Catalyst | (bar) | (° C.) | (h) | n oxalate | V solvent | (Selectivity) |
| A-11 | Co(5%)—Cu(10%)—ZrO₂ | 50 | 200 | 16 | 20 mmol DEO | 50 mL | yld = 57% |
| | (B) | | | | | Ethanol | (selec: 77%) |
| | (4 mmol) | | | | | | |
| | 20 mol % | | | | | | |
| A-12 | Co(5%)—Cu(10%)—SiO₂ | 50 | 200 | 16 | 20 mmol DEO | 50 mL | Yld = 40% |
| | (4 mmol) | | | | | Ethanol | (selec: 32%) |
| | 20 mol % | | | | | | |
| A-13 | Co(5%)—Cu(10%)-γ- | 50 | 200 | 16 | 20 mmol DEO | 50 mL | Yld = 50% |
| | Al₂O₃ | | | | | Ethanol | (selec: 79%) |
| | (4 mmol) | | | | | | |
| | 20 mol % | | | | | | |

### EXAMPLE 10—GENERAL PROCEDURE FOR HETEROGENEOUS CATALYSIS OF THE HYDROGENATION OF DIETHYL OXALATES IN A 1 LITRE REACTOR

In a 1-litre Parr autoclave, equipped with a magnetic stirrer, a heterogeneous catalyst based on copper (40 mmol Cu), diethyl oxalate (200 mmol) and ethanol (400 mL) as a solvent were introduced. The reactor was sealed, and the reaction mixture was purged three times with nitrogen (5 bars), and twice with hydrogen (5 bars).

The autoclave was then pressurized with 50 bars of hydrogen. The reaction medium was then heated to 200° C. for 16 hours or 220° C. for 8 hours.

Once the reaction was complete, the autoclave was brought back to room temperature before being depressurized and purged three times with nitrogen (5 bar).

Ethylene glycol was recovered after purification by vacuum distillation at 120° C./5 mbar.

### EXAMPLE 11. TESTS CARRIED OUT WITH THE COCU/ZRO₂(A) CATALYST IN A 1-LITER REACTOR

Table 5 reports the conditions of the hydrogenation tests of an oxalate, diethyloxalate (DEO) with a CoCu/ZrO₂(A) catalyst carried out in a 1 litre reactor according to example 11, as well as the yield (Yld). The yield of ethylene glycol formed at the end of the hydrogenation step is calculated using GC-MS, mesitylene is used as the internal standard.

TABLE 5
Test conditions of hydrogenation with a CoCu/ZrO₂(A) catalyst
in a 1-litre reactor and the obtained results.

| | | H₂ | T, | Time, | | | |
|---|---|---|---|---|---|---|---|
| Reaction | Catalyst | (bar) | (° C.) | (h) | n oxalate | V solvent | Yield |
| B-1 | Co(5%)—Cu(10%)—ZrO₂ | 70 | 220 | 16 | 200 mmol DEO | in 400 mL | 75% |
| | (4 mmol) | | | | | Ethanol | |
| | 20 mol % | | | | | | |
| B-2 | Co(5%)—Cu(10%)—ZrO₂ | 70 | 220 | 11 | 200 mmol DEO | in 400 mL | 36% |
| | (4 mmol) | | | | | Ethanol | |
| | 20 mol % | | | | | | |

### EXAMPLE 12—GENERAL PROCEDURE FOR CATALYST RECYCLING AFTER HYDROGENATION OF DIALKYL OXALATES

After a dialkyl oxalates hydrogenation reaction, the Co(5%)Cu(10%)/ZrO₂(A) catalyst is separated from the liquid reaction medium by filtration. The catalyst is then washed 3×25 mL of ethanol and centrifuged. The material was then dried at 80° C. for 4 hours before being used again in a dialkyl oxalates hydrogenation reaction under the same conditions as reaction 7 in Table 3.

### EXAMPLE 13—CATALYST RECYCLING TESTS AFTER HYDROGENATION OF DIALKYL OXALATES

Table 6 below reports recycling results according to example 12 respectively.

TABLE 6
Yield and selectivity of a recycled catalyst

| | Cycles | Yield |
|---|---|---|
| C-1 | 1 | 84% |
| | | (95% selectivity) |
| C-2 | 2 | 85% |
| | | (95% selectivity) |
| C-3 | 3 | 65% |
| | | (50% selectivity) |

### EXAMPLE 14—BET ANALYSES OF CU—CO/ZRO₂ CATALYSTS

Table 7 below shows the average specific area, analysed by BET, of Cu(10%)-Co(5%)/ZrO₂ catalysts prepared with various zirconium dioxide supports, after calcination at 600° C. for 2 hours.

TABLE 7
Average specific surface area by BET of
Cu—Co/ZrO₂ catalysts after calcination.

| Compounds | Average specific area (m²/g) |
|---|---|
| ZrO₂ (A) | 6.11 |
| Co(5%)Cu(10%)ZrO₂(A) | 5.44 |
| Co(5%)Cu(10%)ZrO₂(B) | 59.09 |

### EXAMPLE 15: STRUCTURAL ANALYSIS

### A. Phase Analysis

The X-ray powder diffractograms of the materials (catalysts or support) were carried out with a MINIFLEX II diffractometer from Rigaku, whose X-rays radiation was emitted via tube and a copper source (wavelength Kα 1.54 Å).

FIG. 1 shows the RX diffractogram obtained for Co(5%)Cu(10%)/ZrO₂(A) catalyst calcined at 600° C. for 2 hours, having a specific surface area of 5.4 m²/g.

FIG. 2 shows the RX diffractogram obtained for the support used ZrO₂(A) calcined at 600° C. for 2 hours, having a specific surface area of 6.1 m²/g.

FIG. 3 shows the RX diffractogram obtained for Co(5%)Cu(10%)/ZrO₂ (B) catalyst calcined at 600° C. for 2 hours, having a specific surface area of 59 m²/g.

The results of the exploitation of the different diffractograms obtained are presented in tables 8 to 10 below, respectively for:
the Co(5%)Cu(10%)/ZrO₂(A) catalyst in Table 8,
the ZrO₂(A) support in Table 9
the catalyst Co(5%)Cu(10%)/ZrO₂ (B) in Table 10

TABLE 8
Phase analysis in the Co(5%)Cu(10%)/ZrO₂ (A) catalyst

| Sample reference | Crystalline phases identified | Record ICDD |
|---|---|---|
| Co(5%)Cu(10%)ZrO₂(A) | Baddeleyite - ZrO₂ | 00-037-1484 |
| | Copper dioxide - Cu₂O | 03-065-3288 |
| | Copper monoxide - CuO | 01-089-5899 |

TABLE 9
Analysis of the phases in the calcined support ZrO₂(A)

| Sample reference | Crystalline phases identified | Record ICDD |
|---|---|---|
| ZrO₂(A) | Baddeleyite - ZrO₂ | 00-037-1484 |

TABLE 10
Phase analysis in the Co(5%)Cu(10%)/ZrO₂(B) catalyst

| Sample reference | Crystalline phases | Record ICDD |
|---|---|---|
| Co(5%)—Cu(10%)—ZrO₂ (B) | Baddeleyite - ZrO2 | 00-037-1484 |
| (monoclinic phase) | Cobalt oxide - CoO | 01-089-2803 |
| | Cobalt oxide - Co₃O₄ | 01-076-1802 |

The diffractograms show the presence of crystallized phases.

XRD analyses indicate that the crystalline phase of Baddeleyite (ZrO₂) is present in the samples catalysed ZrO₂(A), Co(5%)Cu(10%)/ZrO₂(A) and Co(5%)Cu(10%)/ZrO₂ (B).

The diffractogram of Co(5%)Cu(10%)/ZrO₂(A) sample reveals the presence of two additional crystalline phases of Copper (I) oxide, Cu₂O, and of Copper (II) oxide, CuO with a weak signature.

The diffractogram of Co(5%)Cu(10%)/ZrO₂ (B) sample reveals the presence of two additional crystalline phases of Cobalt oxides, CoO and Co_{3O4} with low intensity.

### B. Crystallinity-Microstructure: Crystallite Size Analysis

### Size of Crystallites

The crystallinity of materials is characterized by the size of the crystallites.

The size of the crystallites was qualitatively estimated in order to compare the different ZrO₂ supports of the catalysts.

The size of the crystallites was evaluated according to the following Scherrer formula:

$$t = \frac{k \cdot λ}{H \cdot cos θ} \begin{matrix}t = crystallite size \\ k = correction factor = 0 , 89 \\ λ = source wavelength \\ H = length at half height of peak \\ θ := diffraction angle\end{matrix}$$

The width at half-height was estimated using ImageJ processing software (developed by the National Institutes of Health).

The calculations of the size of the crystallites from the diffractograms of the catalysts Co(5%)Cu(10%)/ZrO₂(A) and Co(5%)Cu(10%)/ZrO₂ (B), are shown in the following Table 11:

TABLE 11
Crystallite sizes

| | | size of the | |
|---|---|---|---|
| | | crystallites | |
| Reference | | (nm) | |
| Cat A | Pd(2%)Cu(10%)/ZrO₂ (A) | 21.2 | nm |
| | support of Sterm | | |
| Cat B | Pd(2%)Cu(10%)/ZrO₂ (B) | 9.5 | nm |

(monoclinic phase - support of Alfa Aesar)

The ZrO₂ (B) support used to prepare Co(5%)Cu(10%)/ZrO₂ (B) is a commercial ZrO₂ oxide powder from Alfa Aesar, having a pore volume of 0.27 cc/g with a specific surface area of more than 85 m²/g (BET).

Small size values, in particular less than 10 nanometers, indicate a structure with low crystallinity. Indeed, the smaller the crystallites, the broader the diffraction peaks. This effect becomes visible for crystallites with a diameter less than 1 μm.

The results indicate that the catalyst prepared with ZrO₂(A) support of Sterm according to the invention and the catalysts prepared with the ZrO₂ (B) support of Alfa Aesar according to the invention have crystallite sizes of 21 nm and 9 nm, respectively. Thus, Cat A and Cat B catalysts differ in the microstructure of the ZrO₂ support.

In addition to the feature related to the specific surface area, these results show that the supports of the Cat A and Cat B catalysts of the invention are different in their microstructure.

### EXAMPLE 16: MORPHOLOGICAL ANALYSIS

The SEM images in FIGS. 4 to 6 were taken using a Zeiss SEM-FEG scanning microscope with pressure-controlled, enabling to observe materials with little or no conductivity without any specific preparation.

The sample was stabilized on carbon adhesive paper to allow SEM observation. It should therefore be noted that the content of the element carbon can be associated with the use of the latter. The results of the SEM observations and EDX analyses are summarized below.

### Co(5%)Cu(10%)/ZrO₂(A) Catalyst

Observations of the SEM images in FIG. 4 reveal the presence of a population of particles with a rounded morphology. These particles are composed mainly of Zirconium (Zr), Copper (Cu), Oxygen (O), Carbon (C) and Cobalt (Co), with a minor amount of Hafnium (Hf), Rhenium (Re) with traces of Silicon (Si). Quantification was carried out by EDX spectrum analysis over an area of these particles. The results expressed in mass percentage are shown in Table 12.

TABLE 12
Chemical composition of Co(5%)Cu(10%)/ZrO₂(A) catalyst

| | Quantification using the |
|---|---|
| Element | EDX spectrum (by wt %) |
| C | 11.01 |
| O | 17.07 |
| Si | 0.29 |
| Co | 6.55 |
| Cu | 19.43 |
| Zr | 42.91 |
| Hf | 1.34 |
| Re | 1.40 |
| Total | 100.00 |

### Calcined ZrO₂(A) Support

Observations of the SEM images in FIG. 5 reveal the presence of several populations of particles (rounded, spherical and angular morphologies). The majority population has a rounded morphology, similar to that of Co(5%)Cu(10%) ZrO₂ sample. These particles are composed mainly of Zirconium (Zr), Oxygen (O) and Carbon (C) with traces of Rhenium (Re), Hafnium (Hf) and Silicon (Si).

The secondary population, with a spherical morphology, is composed primarily of Zirconium (Zr), Oxygen (O) and Carbon (C), with a minor portion of Hafnium (Hf) and Rhenium (Re) with traces of Silicon (Si).

Quantification was carried out by EDX spectrum analysis on two zones of the particles of the support calcined at 600° C. for two hours. The results expressed in percent by weight are shown in Table 13.

TABLE 13
Chemical composition of the ZrO₂(A) support

| | Quantification by EDX | Quantification by EDX |
|---|---|---|
| Element | spectrum zone 1 (by wt %) | spectrum zone 2 (by wt %) |
| C | 56.88 | 17.29 |
| O | 12.24 | 25.47 |
| Si | 0.21 | 0.67 |
| Zr | 29.15 | 53.94 |
| Hf | 0.70 | 1.33 |
| Re | 0.83 | 1.30 |
| Total | 100.00 | 100.00 |

### Co(5%)Cu(10%)/ZrO₂ (B) Catalyst

Observations of MEB images in FIG. 6 reveal the presence of macroscopic particles with polyhedral morphology consisting of smaller particles with a rounded morphology. The particles are predominantly composed of Zirconium (Zr), Copper (Cu), Cobalt (Co) and Oxygen (O) with a smaller proportion of Carbon (C).

Quantification was carried out by EDX spectrum analysis over an area of these particles. The results expressed in percent by weight are shown in Table 14.

TABLE 14
Chemical composition of catalyst Co(5%)Cu(10%)/ZrO₂(B)

| | Quantification by EDX |
|---|---|
| Element | spectrum (by wt %) |
| C | 3.99 |
| O | 17.817 |
| Co | 11.49 |
| Cu | 27.71 |
| Zr | 39.00 |
| Total | 100.00 |

### EXAMPLE 17: SURFACE ANALYSIS BY XPS

Analyses are carried out using a PHI QUANTES photoemission spectrometer. This instrument is equipped with a monochromate X-ray source (aluminum Kα line) as well as a chromium X-ray source for making Hard XPS, a charge neutralization system for electrical insulating samples and a hemispherical electron analyser.

XPS analyses were performed on the Co(5%)Cu(10%)/ZrO₂(A) catalyst calcined at 600° C. for 2 hours and a sample of the ZrO₂(A) support also calcined at 600° C. for 2 hours.

XPS spectra are shown in FIG. 7.

The quantification of the extreme surface, expressed as a mass percentage, on the two samples is shown in Table 15 below:

TABLE 15
Chemical composition by XPS

| Atom | Co(5%)Cu(10%)ZrO₂ (%) | ZrO₂ (%) |
|---|---|---|
| Oxygen (O) | 34.96 | 39.74 |
| Zirconium (Zr) | 44.53 | 54.11 |
| Cobalt (Co) | 5.71 | / |
| Copper (Cu) | 11.13 | / |

Both samples have a low carbon content, mainly from atmospheric pollution.

Co(5 wt %)Cu(10 wt %) ZrO₂ sample consists of Zirconium (Zr), Oxygen (O), Copper (Cu), Cobalt (Co) with traces of Silicon (Si) and Sodium (Na).

ZrO₂ sample is composed of Zirconium (Zr) and Oxygen (O) with traces of Silicon (Si) and Sodium (Na).

### Determination of Chemical Environments

The results of the Gaussian deconvolutions of spectra are presented in Table 16 below:

TABLE 16
Results of XPS analysis

| | | | 2207-E0108269 | | 2207-E0108270 | |
|---|---|---|---|---|---|---|
| | | | Co5Cu10/ZrO2 | | ZrO2 | |
| | | | Position | Quanti | Position | Quanti |
| C | C1s | CC/CH | 285.0 | 76.4% | 285.0 | 80.1% |
| | C1s | C—O/C═O | 288.6 | 23.6% | 288.4 | 19.9% |
| O | O1s | Oxides | 529.5 | 45.7% | 529.5 | 39.0% |
| | O1s | Oxides (transitional metal) | 531.2 | 54.3% | 531.0 | 61.0% |
| Si | Si2p | SiO2 | 102.0 | 48.6% | 101.8 | 37.7% |
| | Si2p | SiOx/CoO (3s) | 103.3 | 51.4% | 103.0 | 62.3% |
| Na | Na1s | Pollution | 1072.2 | 100.0% | 1072.3 | 100.0% |
| Zr | Zr3d5/2 | ZrO₂ | 181.3 | 28.2% | 181.4 | 72.3% |
| | Zr3d5/2 | ZrO₂ | 182.1 | 19.1% | / | / |
| | Zr3d5/2 | ZrO₂ | 183.6 | 31.2% | 183.5 | 27.7% |
| | Zr3d5/2 | ZrO₂ | 184.6 | 21.6% | / | / |
| Cu | Cu2p3/2 | Cu metal/Cu2O | 932.6 | 23.7% | / | / |
| | Cu2p3/2 | CuO | 934.0 | 76.3% | / | / |
| Co | Co2p3/2 | Co₂O₃/Co(OH)₂ | 779.5 | 31.3% | / | / |
| | Co2p3/2 | | 781.3 | 68.7% | / | / |

### C1s Spectrum:

For all samples, the deconvolution of the C1s carbon spectra presents 2 components:
The majority component at 285.0 eV is characteristic of C—C and C—H bonds. These bonds come from air pollution.
The 288.40 eV component is characteristic of the C—O and C═O bonds. These bonds are also representative of surface pollution.

### O1s Spectrum:

For all samples, the deconvolution of the oxygen O₁s spectra presents 2 components:
The majority component at 531.0 eV is characteristic of oxides in general.
The 531.2 eV component is characteristic of transition metal oxides.

### Si2p Spectrum:

For all samples, the deconvolution of the Si2p silicon spectra presents 2 components:
The majority component at 103.3 eV is characteristic of SiOx silicon oxides.
The 102.0 eV component is characteristic of SiO₂ silicon oxide.

It should be noted that the characteristic component of Cobalt (II) oxide CoO is at the same energy as the component corresponding to SiOx.

### Na1s Spectrum:

For all samples, the deconvolution of the Sodium Nas spectra presents a single component characteristic of air pollution.

### Cu₂p3/2 Spectrum:

For Co(5 wt %)Cu(10 wt %) ZrO₂ sample, the deconvolution of the Copper 2p3/2 spectrum has 2 components:
The majority component at 934.0 eV is characteristic of Copper (II) Oxide CuO.

The two strong satellite components at 943.5 eV and 963.5 eV are characteristic of Cu₂+ ions and therefore of Copper (II) oxide CuO.
The component at 932.6 eV is characteristic of metallic copper or copper (I) oxide Cu₂O.

### Co2p3/2 Spectrum:

For Co(5%)Cu(10%) ZrO₂ sample, the spectrum of Cobalt 2p3/2 presents the characteristic components of Cobalt (III) oxide Co₂O₃ and Cobalt (II) oxide CoO as well as Cobalt (II) hydroxide Co(OH) 2.

High-Resolution XPS Spectrum of Zirconium 3d5/2

XPS analyses reveal for all the samples, the predominant presence of Zirconium oxide (ZrO₂), with traces of Silicon oxides (SiO₂ and SiOx) as well as the presence of surface pollution (Carbon and Sodium).

Co(5%)Cu(10%)/ZrO₂ sample shows the additional presence of copper in two oxidized forms: CuO and Cu₂O. XPS analyses also reveal the presence of Cobalt for this sample in the form of Cobalt hydroxide Co(OH)₂ or Cobalt (II) CoO oxide or Cobalt (III) oxide Co₂O₃.

### EXAMPLE 18-CONTINUOUS FLOW HYDROGENATION PROCEDURE

A solution of diethyl oxalate from 0.05 and 0.5 M in ethanol is pumped into the reactor at a rate from 0.3 to 3 mL/min. Then, the pressure of the system is set between 30 and 100 bar using a back-pressure regulator, and then the reactor is heated to between 15° and 220° C. The reactor is composed of a stainless steel tube in which the catalyst (between 150 and 700 mg) used for the reaction is located. Finally, hydrogen—produced in situ in the equipment or introduced from the outside via a valve—is injected into the system at the desired flow rate between 20 and 100 mLN/min. Optionally the catalyst is first brought into contact with the hydrogen stream before the reagents are introduced. Once the system has been stabilized, the product is collected at the reactor outlet in vials before being analysed by GC-MS.

### EXAMPLE 19—PROCEDURE FOR THE DEGRADATION OF ETHYLENE GLYCOL BY REDUCTION UNDER HYDROGEN IN THE PRESENCE OF CUCO/ZRO₂ CATALYST

In a 450 mL Parr autoclave, equipped with a magnetic stirrer, a heterogeneous catalyst based on copper (4 mmol Cu) and cobalt, ethylene glycol (10 mmol), a base, potassium tert-butoxide (KOtBu) and tetrahydrofuran (THF) (75 mL) as a solvent, were introduced. The reactor was sealed, and the reaction mixture was purged three times with nitrogen (5 bars), and twice with hydrogen (5 bars).

The autoclave was then pressurized with 60 bars of hydrogen. The reaction medium was then brought under stirring at 180 to 200° C. for 15 hours.

Once the reaction was complete, the autoclave was brought back to room temperature before being depressurized and purged three times with nitrogen (5 bar).

The final mixture obtained was diluted and an internal standard was added (mesitylene), to calculate the conversion of ethylene glycol using GC-MS.

The balance of the degradation reaction of ethylene glycol by hydrogenation in the presence of CuCo/ZrO₂ catalyst is as follows:

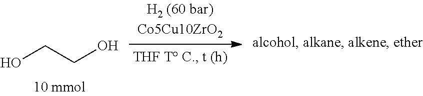

### EXAMPLE 20—TESTS PERFORMED WITH COCU/ZRO₂(A) CATALYST

Table 17 shows the conditions for hydrogenation degradation tests of ethylene glycol in the presence of a CoCu/ZrO₂(A) catalyst prepared according to Example 5. Ethylene glycol conversion is calculated using GC-MS, mesitylene is used as the internal standard.

TABLE 17
Test conditions of hydrogenation of ethylene glycol in the
presence of CoCu/ZrO₂(A) catalyst and the obtained results

| | | | | | H₂ | T, | Time, | |
|---|---|---|---|---|---|---|---|---|
| Reaction | Cat | Base | EG | Solvent | (bar) | (° C.) | (h) | Conversion |
| 1 | Co(5 wt %)—Cu(10 | KOtBu | 10 mmol | THF (75 mL) | 60 | 180 | 15 | 30% |
| | wt %)—ZrO₂ | (4 mmol) | | | | | | |
| | (4 mmol) | 40 mol % | | | | | | |
| | 40 mol % | | | | | | | |
| 2 | Co(5 wt %)—Cu(10 | KOtBu | 10 mmol | THF (75 mL) | 60 | 200 | 15 | 42% |
| | wt %)—ZrO₂ | (4 mmol) | | | | | | |
| | (4 mmol) | 40 mol % | | | | | | |
| | 40 mol % | | | | | | | |
| 3 | Sans | KOtBu | 10 mmol | THF (75 mL) | 60 | 200 | 15 | 1% |
| | catalyseur | (4 mmol) | | | | | | |
| | | 40 mol % | | | | | | |

### EXAMPLE 21: SYNERGISTIC AND PROMOTER EFFECT OF COBALT ON CATALYSTS BASED ON COPPER SUPPORTED BY ZIRCONIUM DIOXIDE IN THE OXALATE HYDROGENATION REACTION FOR THE PREPARATION OF ETHYLENE GLYCOL

Tests with a Mixture

Test CUCOMIX01 was prepared according to the conditions of tests A-2 and A-1 in example 7, with as catalytic material an equimolar mixture of the two monometallic catalysts Co(5%)/ZrO₂ and Cu(10%)/ZrO₂ with a hydrogenation reaction time of 10 hours instead of 16 hours.

Test CUCMIX02 was prepared according to the conditions of test A-7 in Example 7 with the addition of support material (ZrO₂) and with a reaction time of 10 hours instead of 16 hours.

Table 18 below shows the results of substrate conversion rates (oxalate), ethylene glycol yield and selectivity to ethylene glycol production from oxalate hydrogenation tests under similar conditions (temperature, pressure, time) in the presence of:
a monometallic cobalt or nickel supported catalyst (tests A-1, A-2 and A-3) or
a mixture of the two monometallic catalysts (CUCOMIX01 test), or
a mixture of a bimetallic catalyst with an equivalent amount by weight of support (zirconium dioxide) or
a bimetallic catalyst based on copper and cobalt, at a constant copper content, i.e. at a copper content of 10% by total weight of the catalyst (tests A-6 and A-7).

TABLE 18
The results obtained from oxalate conversion, EG yield and EG selectivity of oxalate hydrogenation
tests in the presence of a copper and/or cobalt catalyst on a zirconium dioxide support.

| | | | EG | EG | | |
|---|---|---|---|---|---|---|
| | | Conversion | yield | selectivity | n (Cu) | Time |
| Test | Catalyst | (%) | (%) | (%) | (mmol) | (h) |
| A-2 | Co(5%)—ZrO₂ | 0 | 0 | 0 | 0 | 16 h |
| A-3 | Co(10%)—ZrO₂ | 0 | 0 | 0 | 0 | 16 h |
| A-1 | Cu(10%)—ZrO₂ | 92 | 12 | 13 | 4 | 16 h |
| CUCOMIX01 | 1*Co(5%)/ZrO₂ + 1*Cu(10%)/ZrO₂ | 46 | 0 | 0 | 4 | 10 h |
| CUCOMIX02 | 1*Co(5%)Cu(10%)/ZrO₂ + 1*ZrO₂ | 98 | 42 | 43 | 4 | 10 h |
| A-6 | Co(10%)Cu(10%)—ZrO₂(16 h) | 79 | 56 | 71 | 4 | 16 h |
| A-7 | Co(5%)Cu(10%)—ZrO₂(16 h) | 88 | 84 | 95 | 4 | 16 h |

For the hydrogenation reaction of oxalate to ethylene glycol (EG), the results of the test series in Table 18 and FIG. 8 show that:
1) Monometallic cobalt catalysts supported on zirconium dioxide are inactive. Catalysts with two cobalt contents were tested, namely Co(5%) ZrO₂ and Co(10%) ZrO₂ and gave the same results: cobalt supported on zirconium dioxide does not exhibit catalytic activity after 16 hours of reaction
2) Monometallic copper catalysts supported on zirconium dioxide are active but the ethylene glycol yield is low (12%) even after 16 hours of reaction with the Cu(10%)/ZrO₂ catalyst.
3) The bimetallic catalysts based on copper at a content of 10% and based on cobalt on zirconium dioxide, namely Co(5%)Cu(10%)/ZrO₂ and Co(10%)Cu(10%)/ZrO₂, are more active than the monometallic copper catalyst Cu(10%)/ZrO₂. This alone proves the promoter effect of the presence of cobalt which has been shown to be inactive at these percentages (5% and 10%) in the absence of copper.
4) The mixture of the monometallic catalysts Co(5%)/ZrO₂+Cu(10(%)/ZrO₂ is less active than the bimetallic catalyst Co(5%)Cu(10%) ZrO₂, demonstrating a synergy between cobalt and copper, which would be due to the promoting effect of cobalt which make it possible to modulate the catalytic activity of copper supported on zirconium dioxide.

### EXAMPLE 22: EFFECT OF COBALT CONTENT ON BIMETALLIC CATALYST BASED ON COPPER AND COBALT ON ZIRCONIUM DIOXIDE SUPPORT IN THE HYDROGENATION REACTION OF OXALATE FOR THE PREPARATION OF ETHYLENE GLYCOL

Table 19 below shows the results of substrate conversion rates (oxalate), ethylene glycol yield and selectivity towards ethylene glycol production from oxalate hydrogenation tests under similar conditions (temperature, pressure, time) in the presence of a supported catalyst based on copper at a constant rate of copper, i.e. at a copper content of 10% by total weight of the catalyst, in the absence of cobalt and in the presence of a variable cobalt content from 1% to 10% by total weight of the catalyst.

TABLE 19
Obtained results of oxalate conversion, EG yield and EG selectivity of
oxalate hydrogenation tests in the presence of a catalyst based on copper
with a constant content of 10% in the absence and in the presence of
a cobalt content from 1 to 10% on a zirconium dioxide support.

| | | | EG | EG | Cu | |
|---|---|---|---|---|---|---|
| | | Conversion | yield | selectivity | amount | Time |
| Test | Catalyst | (%) | (%) | (%) | (mmol) | (h) |
| A-1 | Cu(10%)—ZrO₂ | 92 | 12 | 13 | 4 | 16 |
| A-4 | Co(1%)Cu(10%)—ZrO₂ | 117 | 14 | 12 | 4 | 16 |
| A-5 | Co(2%)Cu(10%)—ZrO₂ | 83 | 79 | 95 | 4 | 16 |
| A-7 | Co(5%)Cu(10%)—ZrO₂ | 88 | 84 | 95 | 4 | 16 |
| A-6 | Co(10%)Cu(10%)—ZrO₂ | 79 | 56 | 71 | 4 | 16 |

For the hydrogenation reaction of oxalate to ethylene glycol (EG), the results of the series of tests in Table 19 and FIG. 9 show that:

5) Cobalt as a promoter of the reaction, increases the catalytic activity of CoCu/ZrO₂ catalysts, exhibiting a peak for a percentage of cobalt at 5%, and a decrease in activity when the percentage of cobalt is 10%, namely a typical behavior expected of promoter effects.

It is concluded that cobalt has a promoter function in CuCo/ZrO₂ catalysts.

### EXAMPLE 23: ANALYSIS OF THE CUCO/ZRO₂ CATALYST BY PROGRAMMED TEMPERATURE REDUCTION

### Material and Method

Temperature-Programmed Reduction (PTR) or (TRP) analysis is used to characterize solid materials, including metal oxides (type of oxide, oxide mixture, dispersion on a support). This technique of analysis under a reducing atmosphere is known to the skilled person for characterizing heterogeneous catalysts.

It consists of determining the amount of hydrogen consumed as a function of temperature. It allows to determine the temperatures at which reductions from oxidized to metallic forms occur and possibly the nature of the metal oxides and the ratio between the metal oxides present.

Measurements by temperature-programmed reduction in hydrogen (H₂-TPR) were carried out with a Micromeritics Autochem II 2920 analyzer.

In a typical experiment, 50 mg of sample was pretreated at 200° C. (heating rate=10° C./min) for 30 min under a flow of Helium (He) (30 mL/min). Then, the sample was cooled to 30° C., maintaining the flow of He. The reduction analysis was carried out from 30° C. to 900° C. (heating rate=5° C./min) under a flow of 5 vol. % H₂ in Argon (30 mL/min). The final temperature (900° C.) was maintained for 30 minutes.

The H₂-RTP analysis curve of the Co(5%)Cu(10%)/ZrO₂ catalyst over the temperature range from 100 to 500° C. is shown in FIG. 10. It shows that the reduction peak at low temperature has two maximas at 155° C. and 174° C., which can correspond to the consecutive reduction of CuO to Cu₂O and Cu₂O to Cu, respectively. In addition, the high-temperature reduction peaks, centered at 221° C., could be related to the reduction of Co₃O₄ to CoO and further reduction of Co.

1-30. (canceled)

31. A method of preparation of ethylene glycol comprising:
a hydrogenation step by hydrogen of an oxalate compound to ethylene glycol, in the presence of a supported bimetallic catalyst comprising copper and cobalt on a zirconium dioxide support, of formula CoCu/ZrO₂.

32. The method according to claim 31, wherein:
the hydrogenation step comprises:
bringing into contact:
said oxalate compound,
dihydrogen,
said supported catalyst of formula CoCu/ZrO₂, comprising cobalt and copper on a zirconium dioxide support,
optionally a solvent,
to obtain a reaction medium that may be pressurized,
optionally heating said reaction medium,
to obtain ethylene glycol.

33. The method according to claim 31, wherein said oxalate compound is of the following Formula 1: 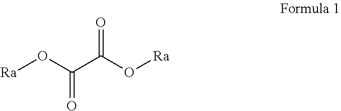
in which Rₐ represents:
a C₁ to C₂₀ linear or branched alkyl group,
a C₃ to C₁₀ cycloalkyl group,
a C₃ to C₂₀ aryl or heteroaryl group, and
a C₅ to C₂₀ alkyl-aryl or alkyl-heretoaryl group.

34. The method according to claim 31, wherein said oxalate compound is selected from dimethyloxalate, diethyloxalate, diphenyloxalate, dibenzyloxalate, isopropyloxalate, and diterbutyloxalate.

35. The method according to claim 31, wherein said catalyst comprises a total content of the bimetallic elements copper and cobalt from 0.5 to 25% by total weight of the catalyst
and in which the ratio by weight between cobalt to copper varies from 1:1 to 1:5,
or wherein the mass composition of cobalt and copper of the catalyst is selected from:
Co(2%)Cu(10%),
Co(5%)Cu(10%),
Co(5%)Cu(5%),
and Co(10%)Cu(10%),

36. The method according to claim 31, wherein said catalyst has a surface area, analysed by BET, from 1 to 250 m²/g or from 1 to 50 m²/g or from 1 to 10 m²/g or about 5 m²/g.

37. The method according to claim 31, wherein the hydrogenation reaction of said oxalate compound by hydrogen to obtain ethylene glycol is selective, with a selectivity greater than 70%,
and/or wherein the hydrogenation reaction of said oxalate compound by hydrogen to obtain ethylene glycol has a yield greater than 70%.

38. The method according to claim 31, wherein said zirconium dioxide support of said CoCu/ZrO₂ catalyst comprises baddeleyite in an amount from 50 to 90% by total weight of the catalyst.

39. The method according to claim 31, wherein the hydrogen is used under a pressure from 2 to 10 MPa,
wherein the reaction medium is heated to a temperature from 100 to 250° C., or from 180 to 220° C., wherein the reaction medium is heated for 5 to 24 hours, or for 8 or 16 hours,
wherein during said heating step of the reaction medium, said supported bimetallic catalyst is activated, said activated catalyst consisting of:
from 80 to 100%, or from 95 to 100%, or 100%, of metallic copper atoms at oxidation degree zero Cu(0) and
from 80 to 100% of cobalt atoms at an oxidation degree greater than zero.

40. A supported bimetallic catalyst comprising copper and cobalt on a zirconium dioxide support, of formula CoCu/ZrO₂,
wherein said catalyst has a surface area, analysed by BET, from 1 to 75 m²/g or from 1 to 50 m²/g.

41. The catalyst according to claim 40, wherein said catalyst has a surface area, analysed by BET, from 1 to 10 m²/g or about 5 m²/g.

42. The catalyst according to claim 40, wherein said zirconium dioxide support comprises a crystalline phase, analysed by X-ray diffraction, crystallized in a monoclinic crystalline system.

43. The catalyst according to claim 42, wherein said crystalline phase represents from 50 to 90% by total weight of the catalyst, or wherein said crystalline phase is baddeleyite.

44. The catalyst according to claim 40, wherein said catalyst comprises a crystalline phase, analysed by X-ray diffraction, comprising a crystallite size from 15 to 100 nm.

45. The catalyst according to claim 40, wherein said catalyst comprises a total content of the bimetallic elements copper and cobalt from 0.5 to 25% by total weight of the catalyst,
or wherein the ratio by weight between cobalt and copper varies from 1:1 to 1:5,
or wherein the mass composition of cobalt and copper of the catalyst is selected from:
Co(2%)Cu(10%),
Co(5%)Cu(10%),
Co(5%)Cu(5%),
and Co(10%)Cu(10%).

46. The catalyst according to claim 40, wherein said catalyst has a molar amount of more than 50% of the element copper at oxidation degree (II) and/or of the element cobalt at oxidation degree (II).

47. The catalyst according to claim 40, wherein said catalyst is in the form of a population of micrometric particles from 1 to 500 μm, or in the form of a population of particles of rounded morphology.

48. The catalyst according to claim 40, wherein said catalyst analysed by temperature-programmed reduction (TPR) carried out in a dihydrogen reducing atmosphere over a temperature range from 30 to 900° C., is characterized by a reduction temperature of metallic copper of oxidation degree (0) in the range from 150 to 250° C.,
at a temperature higher by at least 10° C. than said reduction temperature of metallic copper analysed by TPR,
copper atoms are from 80 to 100%, or 100% in metallic form, and
cobalt atoms are from 80 to 100%, or 100% in oxidized form.

49. The catalyst according to claim 40, wherein said catalyst in reducing atmosphere comprising hydrogen at a pressure from 2 to 10 MPa and at a temperature from 150 to 250° C., or from 180 to 220° C., comprises:
from 80 to 100%, or from 95 to 100%, or 100%, of metallic copper atoms Cu(0), and
from 80 to 100% of cobalt atoms at an oxidation degree greater than zero.

50. A method of preparation of a CoCu/ZrO₂ catalyst according to claim 40, comprising:
a step A of impregnating a cobalt salt and a copper salt, dissolved in an aqueous solution, free of additives and surfactants, in a volume of water from 5 to 10 mL, on a zirconium dioxide support in powder form, with a ratio of solution mass to support mass from 0.6 to 1.0;
to obtain the CoCu/ZrO₂ catalyst in the form of a homogeneous mixture,
said support having
a surface area from 1 to 250 m²/g, or from 1 to 75 m²/g, or from 1 to 50 m²/g, or from 1 to 5 m²/g
and comprising a crystalline phase, analysed by X-ray diffraction,
crystallized in a monoclinic crystalline system,
a step B of drying said homogeneous material, at a temperature from 60 to 100° C., for a period from 10 to 24 hours, to obtain the CoCu/ZrO₂ catalyst in the form of a dry homogeneous mixture, and
an activation step C, comprising calcination under air of said dry homogeneous mixture at a temperature from 200 to 1000° C., for a period from 1 to 15 hours, to obtain said catalyst.
